(19) United States
(12) Patent Application Publication
(10) Pub. No.: US 2024/0087701 A1
(43) Pub. Date: Mar. 14, 2024

(54) SYSTEMS AND METHODS FOR WELLNESS-ENABLED MULTI-RESIDENT COMMUNITY

(71) Applicant: ARCHETYPE Wellness LLC, Charlotte, NC (US)

(72) Inventors: Jaykant R. Patel, Charlotte, NC (US); Rama K. Penta, Charlotte, NC (US)

(21) Appl. No.: 18/463,654

(22) Filed: Sep. 8, 2023

Related U.S. Application Data
(60) Provisional application No. 63/375,095, filed on Sep. 9, 2022.

(51) Int. Cl. G16H 20/00

(52) U.S. Cl. CPC G16H 20/00; H04N 23/66

(57) ABSTRACT

A smart community management and wellness system designed to improve the wellness, lifestyle, and longevity of a community. The smart community management and wellness system provides real-time control of appliances and other electrical devices based on an analysis of an individual's physiological condition using data captured by a wearable device, data inputted or received via a remote device, and/or at least one sensor. The smart community management and wellness system is further operable to monitor the activity of a plurality of users and a plurality of locations and generate at least one recommendation based on user activity and location.

### CROSS-REFERENCE TO RELATED APPLICATIONS

This application claims the benefit and priority to U.S. Provisional Patent Application No. 63/375,095, filed on Sep. 9, 2022, the entire content of which is incorporated by reference herein in its entirety.

### BACKGROUND

### Field of the Invention

The present invention generally relates to systems and methods for controlling multi-user communities and more specifically to systems and methods for controlling multi-user communities based on data captured by wearable devices and other remote devices to provide personalized wellness recommendations and analysis.

### Description of Related Art

Current smart community management systems are designed for a single residence and single use businesses. Additionally, many of the smart community management systems are not self-autonomous as the smart community management systems require input from a user to control appliances. This requires a user to preset settings for multiple appliances via a remote device. Additionally, these user-dependent smart community management systems do not adapt to a user's physiological condition in real-time or over time. Yet another disadvantage is that the current smart building management systems do not support or adapt to a plurality of residents. Additionally, current smart building management systems do not work with non-residents.

Another problem with many smart community systems is the lack of compatibility between Internet of Things (IoT) devices and platforms. Many IoT devices require a specific software platform and only work with other IoT devices compatible with the same specific software platform. This limits the capabilities of a smart community management system and requires users to only use IoT devices compatible with one smart platform. Therefore, there is a need for a smart community management system that is self-autonomous, adaptable based on physiological data, and is configured for multi-resident and multi-use communities.

### BRIEF SUMMARY

The present invention includes a smart community management and wellness system that is operable with IoT devices and systems and non-IoT devices for both residents and non-residents of a community. The smart community management system is designed for lifestyle optimization through the use of physiological monitoring (e.g., biomarkers), habit formation, and personalized wellness recommendations.

The present invention includes a smart community management and wellness system for a multi-resident community configured to provide real-time control of appliances and other electrical devices based on an analysis of physiological conditions using data captured by wearable devices and/or other electrical and remote devices corresponding to a plurality of users. The smart multi-user community management and wellness system is further operable to monitor the activity of a plurality of users and generate at least one recommendation based on user activity.

The subject matter disclosed herein is directed to a wellness-focused smart multi-tenant community management system. The community management system is designed to receive data from a plurality of locations (e.g., a fitness center, and a grocery store) and physiological data captured by wearable devices, remote devices, and sensors to provide real-time analysis and wellness-based recommendations. Advantageously, the present invention is configured to optimize community system settings and provide lifestyle recommendations with a focus on providing and enabling wellness and longevity for the users and residents of the community.

The subject matter disclosed herein is further directed to a smart multi-use community management and wellness system for multi-use environments and a plurality of users. The subject matter disclosed herein is designed to collect data from a plurality of users, and appliances for both residential and commercial activity and provides real-time recommendations, analysis, and control based on the user data and the appliance data at an individual and community level.

In some embodiments, a smart community management and wellness system including at least one remote device including a user interface, at least one image sensor configured to capture an image, and at least one remote server including a software platform including a plurality of dashboards, an analytics engine, and at least one database is disclosed. The at least one remote device, the at least one image sensor, and the at least one remote server are in network communication. The at least one image sensor is operable to capture image data corresponding to at least one user. The user image data is transmitted to the at least one remote device and the at least one remote server. After receiving the user image data, the at least one remote device is operable to display the user image data via the user interface. After receiving the user image data, the analytics engine of the at least one remote server is designed to analyze the user image data to determine at least one physiological condition of the user. Based on the at least one determined physiological condition of the user, the at least one remote server is operable to transmit the at least one determined physiological condition to the at least one remote device. The at least one remote device is operable to display the at least one determined physiological condition. The at least one remote server is further operable to provide at least one wellness recommendation based on the at least one determined physiological condition. The at least one wellness recommendation includes a sleep recommendation, a nutrition recommendation, a mental health recommendation, and/or a fitness recommendation.

In some embodiments, a smart community management and wellness system including at least one remote device including a user interface, at least one controllable electronic device, at least one image sensor configured to capture an image, and at least one remote server including a software platform including a plurality of dashboards, an analytics engine, and at least one database is disclosed. The at least one database includes user data, property data, electronic device data, and health data. The at least one remote device, the at least one image sensor, the at least one controllable electronic device, and the at least one remote server are in network communication. The at least one image sensor is operable to capture image data corresponding to at least one user. The user image data is transmitted to the at least one remote device and the at least one remote server. After receiving the user image data, the at least one remote device is operable to display the user image data via the user interface. After receiving the user image data, the analytics engine of the at least one remote server is designed to analyze the user image data to determine at least one physiological condition of the user. Based on the at least one determined physiological condition of the user, the at least one remote server is operable to transmit the at least one determined physiological condition to the at least one remote device. The at least one remote device is operable to display the at least one determined physiological condition. The at least one remote server is further operable to provide at least one wellness recommendation based on the at least one determined physiological condition. The at least one wellness recommendation includes a sleep recommendation, a nutrition recommendation, a mental health recommendation, and/or a fitness recommendation. The at least one remote server is further operable to transmit an activation command to the at least one controllable electronic device based on the at least one determined physiological condition.

In some embodiments, a smart community management and wellness system including a plurality of remote devices with user interfaces, a plurality of controllable electronic devices, a plurality of wearable devices, and at least one remote server comprising at least one software platform, an analytics engine, and at least one database is disclosed. The plurality of remote devices, the plurality of wearable devices, the plurality of controllable electronic devices, and the at least one remote server are in network communication. Each wearable device of the plurality of wearable devices is designed to capture real-time physiological data of at least one user. Each controllable electronic device of the plurality of controllable electronic devices corresponds to at least one user. The plurality of remote devices, the plurality of controllable electronic devices, and the plurality of wearable devices are designed to transmit data to the at least one software platform. The plurality of wearable devices transmits the real-time physiological data of at least one user to the at least one remote server. The plurality of controllable electronic devices transmits electronic device data. The electronic device data includes a power status and at least one setting corresponding the plurality of controllable electronic devices. The analytics engine is designed to determine at least one physiological condition of at least one user based on the real-time physiological data based on the at least one determined physiological condition of at least one user, the at least one remote server is operable to generate at least one wellness recommendation based on the at least one determined physiological condition. The at least one wellness recommendation includes at least one action to improve the at least one determined physiological condition. The at least one remote server is further operable to transmit an activation command to at least one controllable electronic device of the plurality of controllable electronic devices based on the at least one determined physiological condition and the at least one wellness recommendation.

### BRIEF DESCRIPTION OF THE SEVERAL VIEWS OF THE DRAWINGS

The embodiments illustrated, described, and discussed herein are illustrative of the present invention. As these embodiments of the present invention are described with reference to illustrations, various modifications or adaptations of the methods and or specific structures described may become apparent to those skilled in the art. It will be appreciated that modifications and variations are covered by the above teachings and within the scope of the appended claims without departing from the spirit and intended scope thereof. All such modifications, adaptations, or variations that rely upon the teachings of the present invention, and through which these teachings have advanced the art, are considered to be within the spirit and scope of the present invention. Hence, these descriptions and drawings should not be considered in a limiting sense, as it is understood that the present invention is in no way limited to only the embodiments illustrated.

FIG. 1 illustrates a schematic diagram of a smart multi-use community management and wellness system according to one embodiment of the subject matter disclosed herein.

FIG. 2 illustrates a schematic diagram of a smart community management and wellness system according to one embodiment of the subject matter disclosed herein.

FIG. 3 illustrates a schematic diagram of a smart community management and wellness system according to one embodiment of the present invention.

FIG. 4 illustrates a schematic diagram of a wellness platform of a smart community management wellness system according to one embodiment of the present invention.

FIG. 5 illustrates a profile dashboard of a software platform of a smart community management and wellness system according to one embodiment of the present invention.

FIG. 6 illustrates a wellness dashboard of a software platform of a smart community management and wellness system according to one embodiment of the present invention.

FIG. 7 illustrates a wellness dashboard of a software platform of a smart community management and wellness system according to one embodiment of the present invention.

FIG. 8 illustrates a food preference dashboard of a software platform of a smart community management and wellness system according to one embodiment of the present invention.

FIG. 9 illustrates a profile dashboard of a software platform of a smart community management and wellness system according to one embodiment of the present invention.

FIG. 10 illustrates a notifications dashboard of a software platform of a smart community management and wellness system according to one embodiment of the present invention.

FIG. 11 illustrates a notifications dashboard of a software platform of a smart community management and wellness system according to one embodiment of the present invention.

FIG. 12 illustrates a communications dashboard of a software platform of a smart community management and wellness system according to one embodiment of the present invention.

FIG. 13 illustrates a wellness dashboard of a software platform of a smart community management and wellness system according to one embodiment of the present invention.

FIG. 14 illustrates a nourishment dashboard of a software platform of a smart community management and wellness system according to one embodiment of the present invention.

FIG. 15 illustrates a wellness dashboard of a software platform of a smart community management and wellness system according to one embodiment of the present invention.

FIG. 16 illustrates a sleep wellness dashboard of a software platform of a smart community management and wellness system according to one embodiment of the present invention.

FIG. 17 illustrates a wellness dashboard of a software platform of a smart community management and wellness system according to one embodiment of the present invention.

FIG. 18 illustrates a wellness dashboard of a software platform of a smart community management and wellness system according to one embodiment of the present invention.

FIG. 19 illustrates a nutrition dashboard of a software platform of a smart community management and wellness system according to one embodiment of the present invention.

FIG. 20 illustrates a wellness dashboard of a software platform of a smart community management and wellness system according to one embodiment of the present invention.

FIG. 21 illustrates a wellness dashboard of a software platform of a smart community management and wellness system according to one embodiment of the present invention.

FIG. 22 illustrates a community dashboard of a software platform of a smart community management and wellness system according to one embodiment of the present invention.

FIG. 23 illustrates a guide dashboard of a software platform of a smart community management and wellness system according to one embodiment of the present invention.

FIG. 24 illustrates a profile dashboard of a software platform of a smart community management and wellness system according to one embodiment of the present invention.

FIG. 25 illustrates a medical dashboard of a software platform of a smart community management and wellness system according to one embodiment of the present invention.

FIG. 26 illustrates an amenities dashboard of a software platform of a smart community management and wellness system according to one embodiment of the present invention.

FIG. 27 illustrates a medical dashboard of a software platform of a smart community management and wellness system according to one embodiment of the present invention.

FIG. 28 illustrates a coach dashboard of a software platform of a smart community management and wellness system according to one embodiment of the present invention.

FIG. 29 illustrates a nutrition dashboard of a software platform of a smart community management and wellness system according to one embodiment of the present invention.

FIG. 30 illustrates a wellness dashboard of a software platform of a smart community management and wellness system according to one embodiment of the present invention.

FIG. 31 illustrates a shopping dashboard of a software platform of a smart community management and wellness system according to one embodiment of the present invention.

FIG. 32 illustrates a shopping dashboard of a software platform of a smart community management and wellness system according to one embodiment of the present invention.

FIG. 33 illustrates a nutrition dashboard of a software platform of a smart community management and wellness system according to one embodiment of the present invention.

FIG. 34 illustrates a physiological dashboard of a software platform of a smart community management and wellness system according to one embodiment of the present invention.

FIG. 35 illustrates a screenshot of body map instructions of a software platform of a smart community management and wellness system according to one embodiment of the present invention.

FIG. 36 illustrates a screenshot of body map instructions of a software platform of a smart community management and wellness system according to one embodiment of the present invention.

FIG. 37 illustrates a screenshot of body map instructions of a software platform of a smart community management and wellness system according to one embodiment of the present invention.

FIG. 38 illustrates a screenshot of body map instructions of a software platform of a smart community management and wellness system according to one embodiment of the present invention.

FIG. 39 illustrates a screenshot of body map instructions of a software platform of a smart community management and wellness system according to one embodiment of the present invention.

FIG. 40 illustrates a screenshot of body map instructions of a software platform of a smart community management and wellness system according to one embodiment of the present invention.

FIG. 41 illustrates a screenshot of body map instructions of a software platform of a smart community management and wellness system according to one embodiment of the present invention.

FIG. 42 illustrates a screenshot of body map instructions of a software platform of a smart community management and wellness system according to one embodiment of the present invention.

FIG. 43 illustrates a screenshot of body map instructions of a software platform of a smart community management and wellness system according to one embodiment of the present invention.

FIG. 44 illustrates a screenshot of the results of body mapping of a software platform of a smart community management and wellness system according to one embodiment of the present invention.

FIG. 45 illustrates a smart home dashboard of a software platform of a smart community management and wellness system according to one embodiment of the present invention.

FIG. 46 illustrates a login user interface of a software platform of a smart community management and wellness system according to one embodiment of the present invention.

FIG. 47 illustrates a smart device dashboard of a software platform of a smart community management and wellness system according to one embodiment of the present invention.

FIG. 48 illustrates wellness application programming interface documentation of a software platform of a smart community management and wellness system according to one embodiment of the present invention.

FIG. 49 illustrates building application programming interface documentation of a software platform of a smart community management and wellness system according to one embodiment of the present invention.

FIG. 50 illustrates a schematic diagram of a smart community management and wellness system according to one embodiment of the subject matter disclosed herein.

FIG. 51 illustrates a schematic diagram of a remote server of a smart community management and wellness system according to one embodiment of the subject matter disclosed herein.

FIG. 52 illustrates a schematic diagram of a computer of a smart community management and wellness system according to one embodiment of the subject matter disclosed herein.

FIG. 53 illustrates a schematic diagram of a mobile device of a smart community management and wellness system according to one embodiment of the subject matter disclosed herein.

FIG. 54 illustrates a schematic diagram of a mobile device of a smart community management and wellness system according to one embodiment of the subject matter disclosed herein.

### DETAILED DESCRIPTION

For the purposes of promoting an understanding of the present disclosure, reference will be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “a composite” means at least one composite and can include more than one composite.

Throughout the specification, the terms “about” and/or “approximately” may be used in conjunction with numerical values and/or ranges. The term “about” is understood to mean those values near to a recited value. For example, “about 40 [units]” may mean within +/−25% of 40 (e.g., from 30 to 50), within +/−20%, +/−15%, +/−10%, +/−9%, +/−8%, +/−7%, +/−6%, +/−5%, +/−4%, +/−3%, +/−2%, +/−1%, less than +/−1%, or any other value or range of values therein or there below. Furthermore, the phrases “less than about [a value]” or “greater than about [a value]” should be understood in view of the definition of the term “about” provided herein. The terms “about” and “approximately” may be used interchangeably.

As used herein, the verb “comprise” as is used in this description and in the claims and its conjugations are used in its non-limiting sense to mean that items following the word are included, but items not specifically mentioned are not excluded.

Throughout the specification the word “comprising,” or variations such as “comprises” or “comprising,” will be understood to imply the inclusion of a stated element, integer or step, or group of elements, integers, or steps, but not the exclusion of any other element, integer or step, or group of elements, integers or steps. The present disclosure may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only”, and the like in connection with the recitation of claim elements, or the use of a “negative” limitation.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety.

The subject matter described herein includes a smart community management and wellness system for a multi-resident community and/or individual residences. The smart community management and wellness system is designed for personalized lifestyle optimization including habit formation, coaching recommendations, appliance management, and sleep recommendations based on physiological monitoring (e.g., biomarkers from a digital wellness scan).

The subject matter described herein includes a smart community management and wellness system for a multi-resident community configured to provide real-time control of appliances and other electrical devices based on an analysis of the physiological condition of one or more individuals using data captured by a plurality of wearable devices, a plurality of remote devices, and/or a plurality of sensors. The smart multi-resident community management system is further operable to monitor the activity of a plurality of residents and generate at least one wellness-based recommendation in response to resident activity.

The subject matter disclosed herein is directed to controlling a plurality of appliances based on the physiological condition and activity of at least one user (e.g., a resident, a tenant, a guest). User physiological data, user activity data, and appliance data are analyzed to provide wellness recommendations to users. Settings and/or functions of a plurality of appliances may be changed based on the physiological data. Advantageously, the subject matter described herein encompasses single-family residences (e.g., detached or attached), multi-family residences, multi-tenant residences (e.g., elderly care facilities), and multi-use or mixed-use communities (e.g., a combination of residential and commercial properties).

In some embodiments, a smart community management and wellness system including at least one remote device including a user interface, at least one image sensor configured to capture an image, and at least one remote server including a software platform including a plurality of dashboards, an analytics engine, and at least one database is disclosed. The at least one remote device, the at least one image sensor, and the at least one remote server are in network communication. The at least one image sensor is operable to capture image data corresponding to at least one user. The user image data is transmitted to the at least one remote device and the at least one remote server. After receiving the user image data, the at least one remote device is operable to display the user image data via the user interface. After receiving the user image data, the analytics engine of the at least one remote server is designed to analyze the user image data to determine at least one physiological condition of the user. Based on the at least one determined physiological condition of the user, the at least one remote server is operable to transmit the at least one determined physiological condition to the at least one remote device. The at least one remote device is operable to display the at least one determined physiological condition. The at least one remote server is further operable to provide at least one wellness recommendation based on the at least one determined physiological condition. The at least one wellness recommendation includes a sleep recommendation, a nutrition recommendation, a mental health recommendation, and/or a fitness recommendation.

In some embodiments, the smart community management and wellness system further includes a plurality of controllable electronic devices and a plurality of controllable electric devices. The plurality of controllable electronic devices and the plurality of controllable electric devices are in network communication with the at least one remote server and the at least one remote device. The at least one remote server is operable to transmit an activation command to at least one controllable electronic device of the plurality of controllable electronic devices and/or at least one controllable electric device of the plurality of controllable electric devices based on the at least one determined physiological condition and the at least one wellness recommendation.

In some embodiments, the at least one determined physiological condition includes at least one biomarker. The at least one database further includes historical user data and controllable electronic device data. The historical user data includes biomarker data corresponding to at least one user. The controllable electronic device data includes a power status and at least one setting corresponding to at least one controllable electronic device of the plurality of controllable electronic devices. The analytics engine is further operable to generate at least one controllable electronic device recommendation based on the historical user data and the controllable electronic device data. The at least one controllable electronic device recommendation includes a change of the power status and/or of the at least one setting.

In some embodiments, the at least one determined physiological condition includes at least one biomarker.

In some embodiments, the at least one image sensor is operable to receive at least one command from the at least one remote device. The at least one command includes a request for user image data. In response to the at least one command, the at least one image sensor is activated.

In some embodiments, the smart community management and wellness system further includes at least one wearable device. The at least one wearable device includes a positioning component. The positioning component is operable to track a geographical position of the at least one user. The at least one remote server is operable to modify the at least one wellness recommendation based on user position.

In some embodiments, the at least one database further includes property data and location data for a predetermined geographic area. The property data includes building data and amenities data. The amenities data includes a plurality of amenities in the predetermined geographic area. The at least one wellness recommendation further includes at least one amenity of the plurality of amenities in the predetermined geographic area.

In some embodiments, a smart community management and wellness system including at least one remote device including a user interface, at least one controllable electronic device, at least one image sensor configured to capture an image, and at least one remote server including a software platform including a plurality of dashboards, an analytics engine, and at least one database is disclosed. The at least one database includes user data, property data, electronic device data, and health data. The at least one remote device, the at least one image sensor, the at least one controllable electronic device, and the at least one remote server are in network communication. The at least one image sensor is operable to capture image data corresponding to at least one user. The user image data is transmitted to the at least one remote device and the at least one remote server. After receiving the user image data, the at least one remote device is operable to display the user image data via the user interface. After receiving the user image data, the analytics engine of the at least one remote server is designed to analyze the user image data to determine at least one physiological condition of the user. Based on the at least one determined physiological condition of the user, the at least one remote server is operable to transmit the at least one determined physiological condition to the at least one remote device. The at least one remote device is operable to display the at least one determined physiological condition. The at least one remote server is further operable to provide at least one wellness recommendation based on the at least one determined physiological condition. The at least one wellness recommendation includes a sleep recommendation, a nutrition recommendation, a mental health recommendation, and/or a fitness recommendation. The at least one remote server is further operable to transmit an activation command to the at least one controllable electronic device based on the at least one determined physiological condition.

In some embodiments, the sleep recommendation includes an amount of sleep for the at least one user, the nutrition recommendation includes a food or a liquid for consumption by the at least one user, and the fitness recommendation includes at least one exercise.

In some embodiments, the analytics engine is operable to determine at least one trend based on the at least one determined physiological condition. Based on the at least one trend, the analytics engine is operable to generate at least one habit recommendation. The at least one habit recommendation includes at least one action corresponding to the at least one user for creating a habit.

In some embodiments, the user data includes medical information corresponding to the at least one user, the property data includes at least one residence of the at least one user, the at least one controllable electronic device is located on the residence of the at least one user, the at least one wellness recommendation is based on the user medical information and the at least one determined physiological condition, and the at least one wellness recommendation includes at least one setting corresponding to the at least one controllable electronic device.

In some embodiments, the smart community management and wellness system further includes at least one wearable device in network communication with the at least one remote device and the at least one remote server. The at least one wearable device includes at least one sensor and a positioning component. The at least one sensor is operable to monitor a physiological condition of the at least one user. The positioning component is operable to track a geographical position of the at least one user. The at least one remote server is operable to modify the at least one wellness recommendation based on the user sensor data and the user position.

In some embodiments, the at least one database further includes property data and location data for a predetermined geographic area. The property data includes building data and amenities data. The amenities data includes a plurality of amenities in the predetermined geographic area. The at least one wellness recommendation further includes at least one amenity of the plurality of amenities in the predetermined geographic area.

In some embodiments, a smart community management and wellness system including a plurality of remote devices with user interfaces, a plurality of controllable electronic devices, a plurality of wearable devices, and at least one remote server comprising at least one software platform, an analytics engine, and at least one database is disclosed. The plurality of remote devices, the plurality of wearable devices, the plurality of controllable electronic devices, and the at least one remote server are in network communication. Each wearable device of the plurality of wearable devices is designed to capture real-time physiological data of at least one user. Each controllable electronic device of the plurality of controllable electronic devices corresponds to at least one user. The plurality of remote devices, the plurality of controllable electronic devices, and the plurality of wearable devices are designed to transmit data to the at least one software platform. The plurality of wearable devices transmits the real-time physiological data of at least one user to the at least one remote server. The plurality of controllable electronic devices transmits electronic device data. The electronic device data includes a power status and at least one setting corresponding the plurality of controllable electronic devices. The analytics engine is designed to determine at least one physiological condition of at least one user based on the real-time physiological data based on the at least one determined physiological condition of at least one user, the at least one remote server is operable to generate at least one wellness recommendation based on the at least one determined physiological condition. The at least one wellness recommendation includes at least one action to improve the at least one determined physiological condition. The at least one remote server is further operable to transmit an activation command to at least one controllable electronic device of the plurality of controllable electronic devices based on the at least one determined physiological condition and the at least one wellness recommendation.

In some embodiments, the analytics engine is further configured to determine user activity based on at least one wearable device of the plurality of wearable devices. The analytics engine is further operable to transmit at least one nutrition recommendation to at least one remote device based on the user activity. The at least one nutrition recommendation includes consumption of at least one of protein and/or water.

In some embodiments, each wearable device of the plurality of wearable devices includes at least one sensor and a positioning component. The at least one sensor is operable to monitor the physiological condition of the at least one user. The positioning component is operable to track a geographical position of a user. The at least one remote server is operable to modify the at least one wellness recommendation based on the user sensor data and the user position.

In some embodiments, the at least one database further includes property data and location data for a predetermined geographic area. The property data includes building data and amenities data. The amenities data includes a plurality of amenities in the predetermined geographic area. The at least one wellness recommendation further includes at least one amenity of the plurality of amenities in the predetermined geographic area.

In some embodiments, the at least one software platform includes a messaging platform. The messaging platform is operable for electronic communication between each remote device of the plurality of remote devices.

In some embodiments, the at least one wellness recommendation further includes at least one coaching recommendation. The at least one coaching recommendation includes at least one nutrition coach, at least one fitness coach, and/or at least one lifestyle coach. the software platform is further operable to automatically create an electronic messaging conversation between at least one remote device corresponding to the at least one coaching recommendation and the at least one user.

In some embodiments, each remote device of the plurality of remote devices includes at least one image sensor. The image sensor is designed to capture image data corresponding to at least user. The plurality of remote devices is operable to transmit the user image data to the at least one remote server. The at least one remote server is operable to update the at least one determined physiological condition based on the user image data.

In one embodiment, the smart multi-resident community management system includes a smart speaker (e.g., Amazon Echo®), a smart media device (Google Chromecast®), a smart lighting system (e.g., Phillips® Hue lighting system), a smart heating and cooling system (e.g., Ecobee® Thermostat), a smart security system (e.g., Ring®), a smart access system, smart blinds and shades, a smart switch, and a smart controller. The smart multi-resident community management system is designed for network communication including but not limited to Bluetooth®, Wi-Fi®, and/or ZigBee®. Advantageously, the community management system described herein is configured to communicate with a plurality of IoT devices with and without an internet connection. In one embodiment, the smart multi-resident community management system includes a mesh networking protocol (e.g., Z-wave, Zigbee, Thread). In another embodiment, the smart multi-resident community management system is designed for device-to-device communication, network communication (e.g., Wi-Fi, Ethernet), and cloud communication.

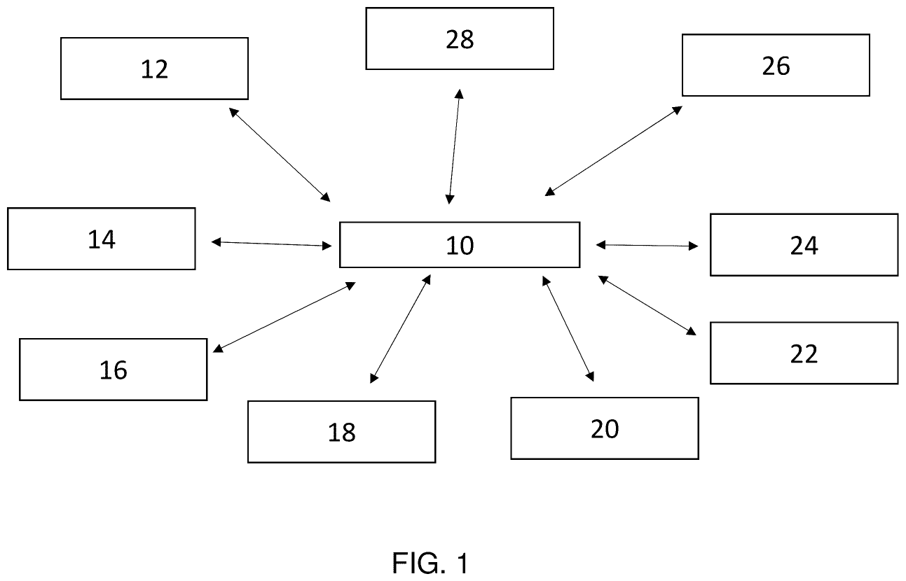

In one embodiment, as shown in FIG. 1, a smart multi-use or mixed-use community management system is disclosed. The community management system described herein is designed for lifestyle optimization for consumer and/or residential uses comprising a plurality of uses and/or residents.

In one embodiment, the smart multi-use community management and wellness system includes a software platform 10 in network communication with one or more facilities that are part of a multi-use community. For example, and not limitation, the one or more facilities include a sports entertainment facility 12 (e.g., a golf course), a recreational facility 14 (e.g., a pool, a gym), a medical facility 16 (e.g., a doctor's office), a nutrition source 18 (e.g., a grocery store, a restaurant, vending machine), at least one residence 20 (e.g., single-family or multi-family dwellings), a security system 22, a religious or spiritual facility 24, an educational institution 26 (e.g., a school), and a traffic system 28. In one embodiment, the community management and wellness system is designed to track user or resident activity via at least one wearable device, at least one remote device, and/or at least one sensor corresponding to each component of the system. The smart community management and wellness system is designed to monitor activity between each component of the smart community management and wellness system to generate automation tasks, recommendations, and commands (e.g., activation commands) based on the user activity.

For example, and not limitation, the community management system may monitor a user's activity and determine that the user played a round of golf in 90-degree heat based on location data and environmental data. The community management system is configured to generate a recommendation to a corresponding user remote device to drink water and to activate the air conditioning system in the user's home. As another example, the community management system may monitor and generate alerts at a community level. For example, and not limitation, based on user medical data transmitted from the medical institution to the software platform, the community management system is designed to determine when there is a disease outbreak. Based on the disease outbreak, the community management system is operable to generate alerts on exposed individuals, sick individuals, and provide lifestyle recommendations (e.g., hydration) to address the disease.

In another embodiment, the community management system includes a software platform with a plurality of dashboards. For example, and not limitation, in one embodiment, the plurality of dashboards includes a property management dashboard (e.g., rent and bill payment, and maintenance requests, a rewards system or program for residents and users of the community), a utility management dashboard (e.g., electrical), a security management dashboard (e.g., gate access to a community), a lifestyle management dashboard (e.g., presents user sleep data and corresponding recommendations), medical management dashboard (e.g., real-time display of user physiological data), and other dashboards corresponding to the monitoring and management of a plurality of users, appliances, and locations within the community.

The community management system is further configured for lifestyle management. For example, and not limitation, the community management system is configured to monitor a user's behavior and identify behavioral changes. Advantageously, the community management system is further operable to generate at least one recommendation and at least one alert based on identified behavioral changes. In another example, and not limitation, the community management system is configured to collect user sleep data via a wearable device, at least one appliance, and/or at least one sensor. Advantageously, the community management system is designed to determine at least one sleep improvement recommendation. For example, and not limitation, if the system determines, based on data from a user's wearable device (for example) that the user is having difficulty sleeping, and the user's medical history (e.g., data from MyChart®) indicates that a user has back problems, then the system may recommend a new mattress and/or automatically order a new mattress designed to target the user's health problems. In another embodiment, the at least one sleep improvement recommendation may include a sound (e.g., white noise) designed to encourage and/or maintain deep sleep. For example, and not limitation, the present invention is designed to track how long a user is in deep sleep while playing a sound at a preset frequency. Advantageously, the present invention is configured to adjust the frequency during a user's sleep to determine an optimal frequency for deep sleep. In an embodiment, the system may adjust the frequency based on real-time information received from the user's wearable tracker in a feedback loop.

The multi-use community management system is further configured to provide digital signage within the community. The digital signage may display information to the residents and others in the community, in some embodiments, based on the data gathered from the users and/or the components within the community.

In one embodiment, the community management system includes a rent management platform and a maintenance platform. Advantageously, the rent management platform enables payment transactions, and the maintenance platform is designed to receive maintenance requests.

The multi-use community management system is further configured to receive real estate data. The real estate data includes zoning regulations for regulating urban design, planning, and development. Advantageously, the present invention is designed to receive real-time zoning updates from government regulatory agencies (e.g., unified development ordinance) and generate at least one alert based on the real-time zoning regulation updates.

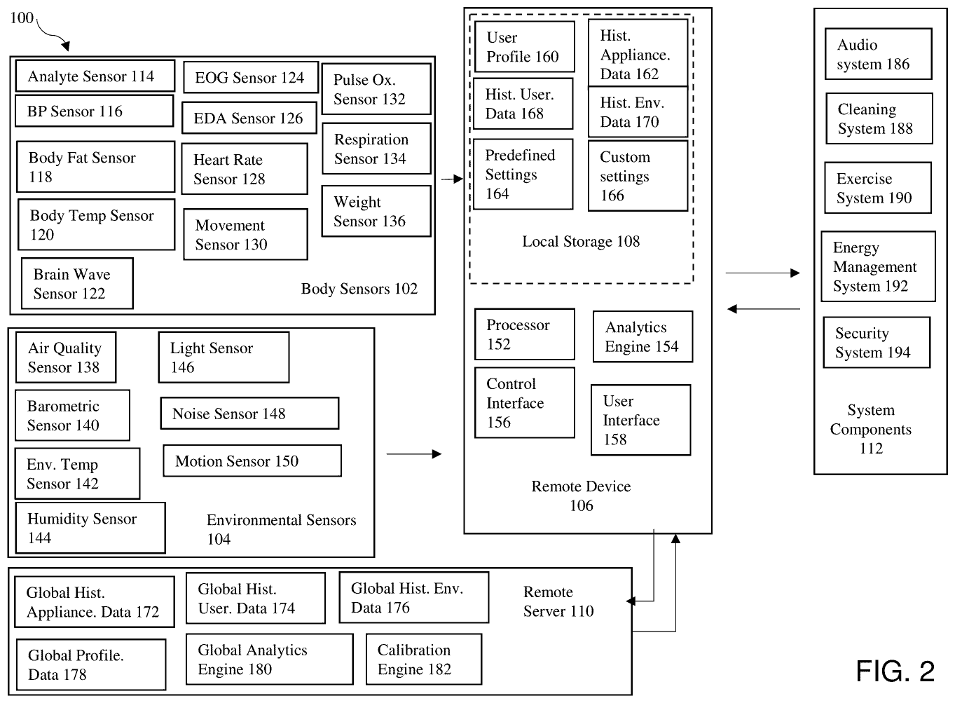

FIG. 2 is a block diagram of one embodiment of the smart multi-resident community management and wellness system. The smart multi-resident community management system 100 includes body sensors 102, environmental sensors 104, a remote device 106 with local storage 108, a remote server 110, and system components 112. Advantageously, the smart multi-resident community management system is designed to capture biomarker data of a user and to provide real-time feedback, analysis, and recommendations based on the biomarker data of the user.

The body sensors 102 include an analyte sensor 114, a blood pressure sensor 116, a body fat sensor 118, a body temperature sensor 120, a brain wave sensor 122, an electrooculography (EOG) sensor 124, an electrodermal activity (EDA) sensor 126, a heart rate sensor 128, a movement sensor 130, a pulse oximeter sensor 132, a respiration sensor 134, and a weight sensor 136.

The present invention is designed to monitor the analyte levels of a user using an analyte sensor 114. Advantageously, the present invention is further operable to change appliance settings (e.g., smart thermostat) and provide recommendations (e.g., decrease sodium intake) based on the analyte sensor data. The analyte sensor 114 monitors levels of an analyte in blood, sweat, or interstitial fluid. In one embodiment, the analyte is glucose, lactate, glutamate, oxygen, sodium, chloride, potassium, calcium, ammonium, copper, magnesium, iron, zinc, creatinine, uric acid, oxalic acid, urea, ethanol, an amino acid, a hormone (e.g., cortisol, melatonin), a steroid, a neurotransmitter, a catecholamine, a cytokine, and/or an interleukin (e.g., IL-6). The analyte sensor 114 is preferably non-invasive. In one embodiment, the analyte sensor 114 is incorporated into a wearable device. Alternatively, or additionally, the smart community management and wellness system is operable to capture image data via a remote device corresponding to blood, sweat, or an interstitial flood and to determine an analyte level based on the captured image data.

In one embodiment, the blood pressure (BP) sensor 116 is preferably wireless. Alternatively, the blood pressure sensor 116 estimates the blood pressure without an inflatable cuff. For example, and not limitation, the blood pressure sensor includes a sphygmomanometer. In one embodiment, the blood pressure sensor 116 is incorporated into a wearable device. Advantageously, the present invention is designed to correlate the blood pressure data with appliance data and environment data. For example, and not limitation, when the present invention detects that at least one user (e.g., resident, tenant, guest) has high blood pressure or has rapidly increasing blood pressure levels, then the present invention is designed to lower the temperature of a corresponding apartment to cool the tenant.

The body fat sensor 118 is preferably a bioelectrical impedance device. In one embodiment, the body fat sensor 118 is incorporated into a smart scale (e.g., Fitbit® Aria®, Nokia® Body+, Garmin® Index™, Under Armour® Scale, Pivotal Living® Smart Scale, iHealth® Core). Alternatively, or additionally, the smart community management and wellness system is operable to capture image data via a remote device corresponding the physique of a user and to determine a body fat percentage based on the captured image data. Advantageously, the present invention is designed to combine the body fat sensor data with appliance data and environment data. For example, and not limitation, the present invention is configured to determine whether a user has an unhealthy increase in body fat (e.g., a user's body fat percentage exceeds 32%) and to generate at least one recommendation to a remote device to reduce caloric intake. Additionally, in one embodiment, the present invention is further operable to transmit an order to a grocery store corresponding to the multi-resident community for low-fat food.

The body temperature sensor 120 measures core body temperature and/or skin temperature in real-time. The body temperature sensor 120 includes but is not limited to a thermistor, an infrared sensor, or a thermal flux sensor. In one embodiment, the body temperature sensor 120 is incorporated into a wearable device (e.g., a watch). Alternatively, or additionally, the smart community management and wellness system is operable to capture image data via a remote device corresponding to the physique of a user and to determine a body fat percentage based on the captured image data. The body temperature sensor 120 is preferably wireless. The present invention is designed to correlate the body temperature data with the appliance data and environmental data. For example, and not limitation, the present invention is designed to determine whether a user's body temperature is rising based on the body temperature sensor. Advantageously, the present invention is operable to transmit a command to a smart thermostat to decrease the temperature in an apartment when a user's body temperature is increasing.

The brain wave sensor 122 is preferably an electroencephalogram (EEG) with at least one channel. In a preferred embodiment, the EEG has at least two channels. Multiple channels provide higher resolution data. The frequencies in EEG data indicate particular brain states. The brain wave sensor 122 is preferably operable to detect delta, theta, alpha, beta, and gamma frequencies. In another embodiment, the brain wave sensor 122 is operable to identify cognitive and emotional metrics, including focus, stress, excitement, relaxation, interest, and/or engagement. In yet another embodiment, the brain wave sensor 122 is operable to identify cognitive states that reflect the overall level of engagement, attention, focus, and/or workload that reflects cognitive processes (e.g., working memory, problem-solving, analytical reasoning). For example, and not limitation, the present invention is operable to determine a tenant's level of engagement with a television. Advantageously, the present invention is operable to power off the television if the user's level of engagement is low (e.g., the user is asleep). Alternatively, or additionally, the smart community management and wellness system is operable to capture image data via a remote device corresponding the positioning and activity of a user during an activity (e.g., sleep) and to determine a level of brain activity based on the captured image data.

The electrooculography (EOG) sensor 124 measures the corneo-retinal standing potential that exists between the front and the back of the eye. In one embodiment, the present invention further includes an eye tracker that is operable to track a user's eye's position and movement data in real-time. For example, and not limitation, the eye data is used to monitor a user's sleep cycle and/or engagement with a television. Advantageously, the present invention is operable to correlate a user's physiological data and appliance data with the eye data in real-time to determine a user's activity.

The electrodermal activity sensor 126 measures sympathetic nervous system activity. In one embodiment, the electrodermal activity sensor 126 is incorporated into a wearable device. Alternatively, or additionally, the smart community management and wellness system is operable to capture image data via a remote device corresponding the skin of a user and to determine electrodermal activity based on the captured image data.

The heart rate sensor 128 is preferably incorporated into the at least one wearable device. The heart rate is determined using electrocardiography, pulse oximetry, ballistocardiography, or seismocardiography. In one embodiment, the heart rate sensor 128 measures heart rate variability (HRV). HRV is a measurement of the variation in time intervals between heartbeats. Advantageously, the present invention is operable to determine a user's activity based on the heart rate sensor. Alternatively, or additionally, the smart community management and wellness system is operable to capture image data via a remote device corresponding the physique and skin of a user and to determine a heart rate and the heart rate variability of a user based on the captured image data. For example, and not limitation, the present invention is configured to determine that a user is undergoing intense cardio exercise based on the heart rate data. The present invention is operable to transmit a command to lower the temperature to a smart thermostat or another electric device operable to control the air conditioning of a smart community and/or apartment. The smart community management and wellness system is further operable to correlate the heart rate data with other physiological data. For example, and not limitation, the smart community management and wellness system is operable to correlate the heart rate data with changes in body temperature, environment data, and movement data.

The movement sensor 130 includes an accelerometer and/or a gyroscope. In one embodiment, the accelerometer and/or the gyroscope are incorporated into at least one wearable device. In another embodiment, the accelerometer and/or the gyroscope are incorporated into a smartphone. In an alternative embodiment, the movement sensor 130 is a non-contact sensor. In one embodiment, the movement sensor 130 is at least one piezoelectric sensor. In another embodiment, the movement sensor 130 is a pyroelectric infrared sensor (i.e., a “passive” infrared sensor). Advantageously, the present invention is configured to transmit commands to appliances and other electronic devices based on the movement data. For example, and not limitation, the present invention is designed to transmit a command to a smart lighting appliance to turn on when the motion sensor detects a user has entered an apartment. The present invention is further configured to determine when a user has left the apartment and transmit a command to the smart lighting appliance to turn off the lights.

The pulse oximeter sensor 132 monitors oxygen saturation. In one embodiment, the pulse oximeter sensor 132 is worn on a finger, a toe, or an ear. The pulse oximeter sensor 132 is preferably wireless. Alternatively, the pulse oximeter sensor 132 is wired. Alternatively, or additionally, the smart community management and wellness system is operable to capture image data via a remote device corresponding to the physique and/or breath of a user and to determine a level of oxygen saturation of a user based on the captured image data.

The respiration sensor 134 measures a respiratory rate. In one embodiment, the respiration sensor 712 is incorporated into a wearable device (e.g., a chest strap). Alternatively, the respiratory rate is estimated from an electrocardiogram, a photoplethysmogram (e.g., a pulse oximeter), and/or an accelerometer. In yet another embodiment, the respiratory sensor 712 uses a non-contact motion biomotion sensor to monitor respiration. Alternatively, or additionally, the smart community management and wellness system is operable to capture image data via a remote device corresponding to the physique and/or breath of a user and to determine a level of oxygen saturation of a user based on the captured image data.

The weight sensor 136 is preferably a smart scale (e.g., Fitbit® Aria®, Nokia® Body+, Garmin® Index™, Under Armour® Scale, Pivotal Living® Smart Scale, iHealth® Core). In another embodiment, a body mass index (BMI) of the user is calculated using the body weight of the user and the height of the user as measured by the at least one pressure sensor. Alternatively, or additionally, the smart community management and wellness system is operable to capture image data via a remote device corresponding to the physique a user and to determine the weight of the user based on the captured image data. In one embodiment, the present invention is designed to monitor the weight sensor of a user and to issue commands to at least one fitness machine to adjust a setting to target fat burning.

The environmental sensors 104 include an air quality sensor 138, a barometric sensor 140, an environmental temperature sensor 142, a humidity sensor 144, a light sensor 146, a noise sensor 148, and a motion sensor 150.

In one embodiment, the present invention includes a plurality of each environmental sensors and is incorporated into a building automation system (e.g., Amazon® Alexa®, Apple® HomeKit™, Google® Home™, IF This Then That® (IFTTT®), Nest®). Alternatively, environmental sensors 104 are incorporated into a smartphone or tablet. In one embodiment, the noise sensor 228 is a microphone. In one embodiment, the air quality sensor 230 measures carbon monoxide, carbon dioxide, nitrogen dioxide, sulfur dioxide, particulates, and/or volatile organic compounds (VOCs). Advantageously, the claimed invention is designed to correlate the environment data with the body sensors data and appliance data.

The remote device 106 is preferably a smartphone or a tablet. Alternatively, the remote device 106 is a laptop or a desktop computer. The remote device 106 includes a processor 152, an analytics engine 154, a control interface 156, and a user interface 158. The remote device 106 accepts data input from the body sensors 102 and/or the environmental sensors 104. The remote device also accepts data input from the remote server 110. The remote device 106 stores data in a local storage 108.

The local storage 108 on the remote device 106 includes a user profile 160, historical appliance data 162, predefined settings 164, custom settings 166, historical user data 168, and historical environmental data 170. The user profile 160 stores appliance preferences and physiological information about the user, including but not limited to, apartment temperature, the brightness of lighting, age, weight, height, body fat, body mass, torso size, hip size, gender, medical history (e.g., sleep conditions, medications, diseases), fitness (e.g., fitness level, fitness activities), sleep goals, nutrition goals, dietary restrictions & preferences, stress level, and/or occupational information (e.g., occupation, shift information). The medical history includes caffeine consumption, alcohol consumption, tobacco consumption, use of prescription sleep aids and/or other medications, blood pressure, restless leg syndrome, narcolepsy, headaches, heart disease, sleep apnea, depression, stroke, diabetes, allergies, nutrition plans (e.g., medical nutrition therapy, medically tailored meals), insomnia, anxiety or post-traumatic stress disorder (PTSD), and/or neurological disorders.

In one embodiment, the weight of the user is automatically uploaded to the local storage from a third-party application. In one embodiment, the third-party application obtains the information from a smart scale (e.g., Fitbit® Aria®, Nokia® Body+™ Garmin® Index™ Under Armour® Scale, Pivotal Living® Smart Scale, iHealth® Core). Alternatively, or additionally, the weight of a user is uploaded via a remote device designed to capture an image of a user.

The historical user data 168 includes information gathered from the body sensors 102. This includes information from the analyte sensor 114, the blood pressure sensor 116, the body fat sensor 118, the body temperature sensor 120, the brain wave sensor 122, the electrooculography (EOG) sensor 124, the electrodermal activity (EDA) sensor 126, the heart rate sensor 128, the movement sensor 130, the pulse oximeter sensor 132, the respiration sensor 134, and the weight sensor 136. Alternatively, or additionally, the historical user data 168 includes information gathered from a remote device designed to capture an image of a user and to determine one or more physiological conditions of a user based on the captured image data.

The historical environmental data 170 includes information gathered from the environmental sensors 104. This includes information from the environmental sensors 104 include information from the air quality sensor 138, the barometric sensor 140, the environmental temperature sensor 142, the humidity sensor 144, the light sensor 146, the noise sensor 148, and the motion sensor 150.

The remote server 110 includes global historical appliance data 172, global historical user data 174, global historical environmental data 176, global profile data 178, a global analytics engine 180, and a calibration engine 182. The global historical appliance data 172, the global historical user data 174, the global historical environmental data 176, and the global profile data 178 include data from a plurality of multi-resident communities.

The remote server 110 is operable to store physiological data, environmental data, user profile data, and appliance data. The physiological data includes data captured by the plurality of physiological sensors. The environmental data includes data captured by the plurality of environment sensors. The appliance data includes data capture by a plurality of remote devices and electrical devices in a multi-user community. The plurality of physiological sensors, the plurality of environment sensors, and the plurality of remote devices are operable to transmit the captured data to the remote server in real-time. The user profile data includes appliance data corresponding to a user's residence.

The global analytics engine 180 is designed to receive the real-time physiological data, environment data, appliance data, and the user profile data. The global analytics engine 180 is configured to evaluate the condition of a user based on the real-time physiological data, environment data, appliance data, and the user profile data. Additionally, the global analytics engine 180 is further operable to generate at least one recommendation based on the received data. For example, and not limitation, the global analytics engine is operable to analyze the energy usage for an apartment. The global analytics engine is further operable to compare the energy usage to other apartments with similar appliances. Advantageously, the global analytics engine is operable to determine whether appliances are functioning properly based on the energy usage. When the global analytics engine determines that the appliances are not functioning properly, the system is configured to order a new appliance and/or order maintenance.

Yet another advantage of the global analytics engine 180 is determining whether a user is at risk while using an exercise machine and generating an alert or command based on the risk. For example, and not limitation, the global analytics engine is operable to compare the real-time physiological data to the medical history of a user. The global analytics engine is designed to detect that a user's heart rate is above a threshold for a user profile. The threshold is based on the medical history of the user. For example, and not limitation, the analytics engine is configured to generate a threshold of about 40 beats per minute above a user's resting heart rate when a user's medical history indicates that the user recently underwent surgery. Next, the global analytics engine is operable to transmit an alert to a wearable device corresponding to the user and/or the exercise machine. The alert indicates that the heart rate of a user is exceeding the threshold. Advantageously, the remote server is operable to issue a command to change at least one setting for the exercise device to address the risk. The at least one setting includes a resistance, a speed, or a power setting. The remote server is further operable to generate an alert for the user to contact a medical professional based on the real-time physiological data.

The global profile engine 178 is designed to create and update a user profile based on the real-time physiological data, appliance data, and data received from the analytics engine. For example, and not limitation, the profile engine is operable to receive information that a user has frequently set a thermostat at the lowest setting during the summer while leaving their window open. The profile engine is further operable to compare a plurality of users with similar thermostat settings and generate a recommendation based on the plurality of users. For example, and not limitation, the profile engine is operable to compare the energy consumption profiles for other users with similar temperature settings and issue at least one command (e.g., close window) based on the comparison to other energy consumption profiles.

The calibration engine 182 is configured to design appliance settings based on a user profile. For example, and not limitation, the calibration engine is configured to determine that a user leaves their apartment from 8 a.m. to 5 p.m. every day. The calibration engine is configured to alter the settings of a smart thermostat to match the activity of a user. For example, and not limitation, to match the user's activity, the calibration engine is configured to reduce the activity of an air conditioner during the hours of 8 a.m. and 5 p.m.

In one embodiment, the system components 112 include an audio management system 186, a cleaning system 188, an exercise system 190, an energy management system 192, and/or a security system 198. For example, and not limitation, the smart community management and wellness system includes a software platform designed to monitor and control each system component. The software platform is in network communication with at least one remote device corresponding to the user and is operable to generate a control interface with a user interface of the remote device. Advantageously, the software platform is operable to receive a command, a request, and/or data corresponding to at least one system component via the user interface of the user remote device.

For example, and not limitation, in one embodiment, the audio system 186 includes a plurality of speakers. Advantageously, the present invention is designed to control the audio system based on the physiological data. For example, and not limitation, if the software platform determines that a plurality of users have elevated stress levels and blood pressures, the system is designed to transmit a command to play calming music.

The cleaning system includes a plurality of cleaning appliances (e.g., a vacuum robot) and works in combination with the environment data and physiological data. For example, and not limitation, in one embodiment, the present invention is operable to determine that a spill occurred in a room and/or hallway based on the environment data and/or user motion data. Advantageously, the present invention is operable to send a command to a vacuum robot to clean up the area affected by the spill.

The exercise system 190 includes a plurality of exercise machines operable for network communication. Advantageously, the present invention is operable to correlate the physiological data with the plurality of exercise machines to design workouts based on the user's physiological condition and/or physiological goals. For example, and not limitation, if a user has a heart rate that exceeds a threshold for a heart rate during a workout, then the present invention is designed to transmit a command to a corresponding exercise machine to lower the intensity until a user's heart rate has returned to a safe range. In yet another embodiment, the exercise system is designed to monitor the activity (e.g., steps and/or exercise) of a user via at least one wearable device (e.g., Fitbit®). For example, and not limitation, the exercise system is designed to monitor a number of steps, types of workouts (e.g., cardio), oxygen consumption, heart rate variability, caloric intake, number of calories burned, and injuries. Alternatively, the exercise system is designed to monitor a user's activity without a wearable device. For example, and not limitation, in one embodiment, the smart community management and wellness system includes a fitness center with a plurality of exercise devices. The smart community management and wellness system is designed to capture physiological data of users of the plurality of exercise devices with and without a wearable device. Advantageously, this enables the present invention to monitor and analyze users of the smart community management and wellness system in real-time and provide wellness-based recommendations based on the physiological data.

The energy management system 192 is designed to manage the power usage of a plurality of appliances. For example, and not limitation, in one embodiment, the energy management system includes a smart thermostat, an air conditioner, smart blinds, smart lighting, fans, and other similar devices. The present invention is designed to control each appliance of the plurality of appliances based on user activity, user physiological data, and environment data. For example, and not limitation, the present invention is operable to determine that a user is not home based on motion activity. Advantageously, the present invention is operable to turn off all lights and reduce the activity of the air conditioner based on the lack of motion.

In one embodiment, the security system 198 includes a camera, a motion detector, a speaker, a microphone, an alarm, and a doorbell. The security system is designed to manage access control to a multi-user community and each residence inside of the multi-user community. For example, and not limitation, the present invention is operable to determine that a user is approaching based on a wearable device attached to a user (e.g., global positioning system and/or component) and deactivate a door lock. In another embodiment, the security system is configured for voice recognition and voice identification.

The body sensors 102, the environmental sensors 104, the remote device 106 with local storage 108, the remote server 110, and the system components 112 are designed to connect directly (e.g., Universal Serial Bus (USB) or equivalent) or wirelessly (e.g., Bluetooth®, Wi-Fi®, ZigBee®) through systems designed to exchange data between various data collection sources. In a preferred embodiment, the body sensors 102, the environmental sensors 104, the remote device 106 with local storage 108, and the remote server 110 communicate wirelessly through Bluetooth®. Advantageously, Bluetooth® emits lower electromagnetic fields (EMFs) than Wi-Fi® and cellular signals.

In one embodiment, the present invention further includes an artificial intelligence engine operable to receive real-time or near real-time data from the body sensors 102, the environment sensors 104, and the system components 112. For example, and not limitation, in one embodiment, the artificial intelligence engine includes at least one machine learning algorithm. In one embodiment, at least one remote device includes the artificial intelligence engine. The at least one remote device includes a cellphone, a laptop, a tablet, a desktop computer, and other remote devices. The artificial intelligence engine includes a visualization component. The visualization component is operable to display the captured and analyzed real-time or near real-time data. The visualization tool is operable to generate and display bar graphs, line graphs, circle graphs, histograms, mosaic charts, a spider chart, a flow chart, a control chart, a waterfall chart, a scatter plot, anatomical diagrams, and other graphical displays to illustrate the captured data.

The artificial intelligence engine is operable to generate a user health score based on the received physiological data and an appliance health score based on appliance data. The at least one remote device is operable to receive user input for physiological user information. The artificial intelligence engine is operable to generate a user's real-time or near real-time user health score. Additionally, the artificial intelligence engine is operable to correlate the real-time or near real-time user health data with the appliance data and environment data. This improves the real-time or near real-time analysis and allows for the present invention to generate real-time or near real-time alerts if the present invention determines that system components and/or users are at risk. The artificial intelligence engine is operable to use historical data to provide prompts and/or questions to a user to improve analysis. Additionally, the artificial intelligence engine is operable to provide user-specific questions based on user's physiological data. Advantageously, the present invention is operable to compare appliance data and body sensor data for at least one user to previous appliance data for other communities and previous sensor data for other users.

In yet another embodiment, the artificial intelligence system is operable to generate a real-time or near real-time alert based on the real-time or near real-time physiological data, environment data, and/or appliance data. The at least one alert includes changes in environmental conditions (ex. room temperature), changes in body data (e.g., body temperature), changes in appliance activity (e.g., air conditioner stop functioning), and other similar alerts relating to the management of a multi-resident community. Additionally, the present invention is configured to provide a recommendation based on the alert. By way of example and not limitation, if the artificial intelligence system determines that the external temperature is increasing, the present invention is operable to send a command to lower blinds to maintain temperature. The present invention is further operable to automatically contact a third-party based on the physiological data and the appliance data. For example, and not limitation, the present invention is operable to detect the efficiency of at least one system component is declining and to request maintenance on the appliance. Advantageously, this allows for a self-autonomous system for managing a multi-resident community.

In one embodiment, the present invention is designed for wellness-based gamification and incentives. For example, and not limitation, the present invention includes a gamification system designed to offer health and wellness initiatives. The gamification system is operable to track and display the progress of a plurality of users for a plurality of wellness-based objectives and incentives. The wellness-based objectives and initiatives include, but are not limited to, participating in community activities (e.g., a road race or walking program), fitness goals (e.g., walking a predetermined distance), and nutritional goals (e.g., eating a predetermined amount of protein). In one embodiment, the gamification system further includes a point system based on user activity and physiological data. For example, and not limitation, the present invention is designed to award points to a user profile based on user activity compared to wellness-based objectives and/or at least one wellness recommendation. In one embodiment, the system is configured to display points accumulation for a plurality of users to encourage community competition. In another embodiment, the present invention is operable for the transfer of accumulated points for at least one reward (e.g., a gift card or a free item at a grocery store).

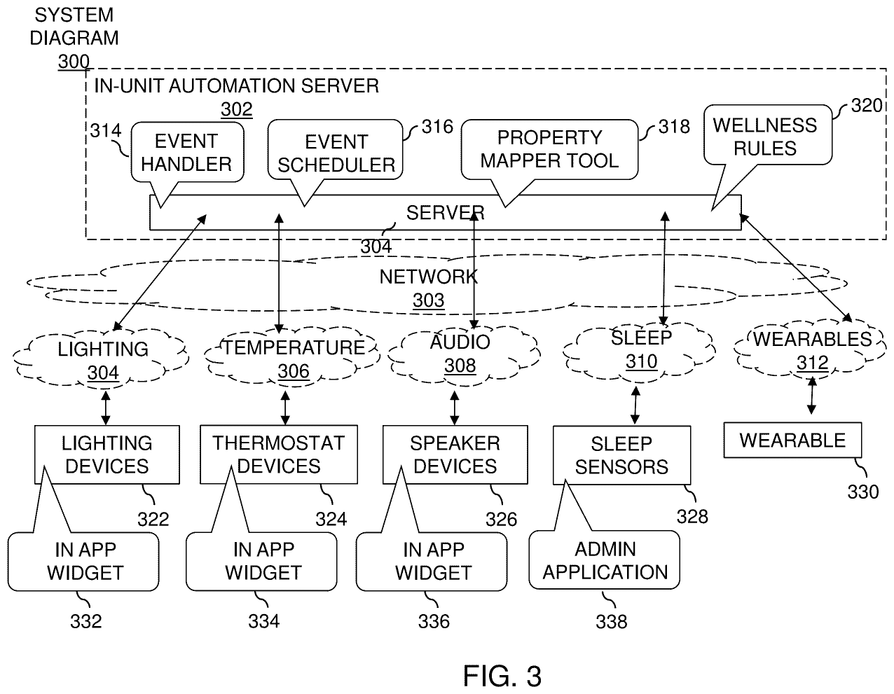

FIG. 3 illustrates a schematic diagram of a smart community management and wellness system according to one embodiment of the present invention. The smart community management and wellness system 300 includes a server 302, a lighting management component 304, a temperature management component 306, an audio management component 308, a sleep management component 310, and a wearable management component 312. The remote server includes an event handler component 314, an event scheduler component 316, a property mapper tool 318, and a wellness rules component 320.

The wellness rules component 320 includes wellness standards and rules corresponding to a user and/or a community. For example, and not limitation, the wellness rules include a sleep routine (e.g., getting ready an hour before an expected bedtime for a suggested eight hours of sleep.) The property mapper tool 318 is designed to monitor appliance data and a location of a user within a property. For example and not limitation, the property manager tool is operable to determine when a user is in the user's bedroom. The event scheduler 316 and event handler 314 work in tandem with each other and the property mapper tool 319 and wellness rules 320. The event scheduler is designed to activate appliances based on the personal wellness rules corresponding to a user. The event handler is designed to control one or more appliances based on the event scheduler. For further example, and not limitation, the smart community management and wellness system is operable to capture user (e.g., resident) usage and user behavior related to a property, amenity, facility and/or activity. For example, and not limitation, the smart community management and wellness system is operable to determine that a facility (e.g., gym) has low user activity. Advantageously, the smart community management and wellness system is designed to transmit the facility data to at least one user (e.g., property manager) and to provide at least one event recommendation and/or at least one operation recommendation. For further example, and not limitation, the at least one event recommendation includes a date and a type of activity to increase user activity. The at least one event recommendation includes an activities class (e.g., yoga), an organized event (e.g., basketball tournament), and other similar events designs to improve activity and engagement. The at least one operation recommendation includes but is not limited to operating hours, cleaning scheduling, maintenance schedule, and similar functions for operating and maintaining a facility. For further example, the smart community management and wellness system is operable to determine when user activity is lowest and to send a notification to a property manager remote device with a recommended time cleaning. Alternatively, or additionally, the smart community management and wellness system is operable to automatically schedule a third-party service provider/or to transmit a command to a controllable electronic device (e.g., IoT vacuum) to clean a facility during the low user activity.

The lighting management component is in network communication with a plurality of lighting devices within a corresponding residence and/or community. For example, and not limitation, the lighting component includes a lamp, lightbulbs, and other similar lighting devices. The lighting temperature component is in network communication with a plurality of temperature control devices within a corresponding residence and/or community. For example, and not limitation, the temperature control devices include a thermostat, a fan, a heating, ventilation, and air conditioning (HVAC) system, and other similar devices and systems. The audio management component is in network communication with a plurality of audio devices including but not limited to radio, speakers, stereos, televisions, and other devices with audio components. The sleep management component is in network communication and is operable to control a plurality of sleep related devices. For example, and not limitation, the sleep related devices includes mattress, beds, bed stands, blinds, and other sleep related devices. The wearable management device component is in network communication and operable to monitor and control a plurality of wearable devices. The plurality of wearable devices include, but are not limited to, watches, headsets, jewelry, glasses, wearable physiological monitoring devices, clothing, and other wearable devices. The lighting management component, the temperature management component, the audio management component, the sleep management component, and the wearable management component are designed to capture real-time data corresponding to the plurality of lighting devices, the plurality of temperature devices, the plurality of audio devices, the plurality of sleep devices, and the plurality of wearable devices. Further, the remote server includes at least one software platform in network communication with at least one remote device. The software platform designed to display the real-time data and historical data to the plurality of lighting devices, the plurality of temperature devices, the plurality of audio devices, the plurality of sleep devices, and the plurality of wearable devices via a user interface of the at least one remote device.

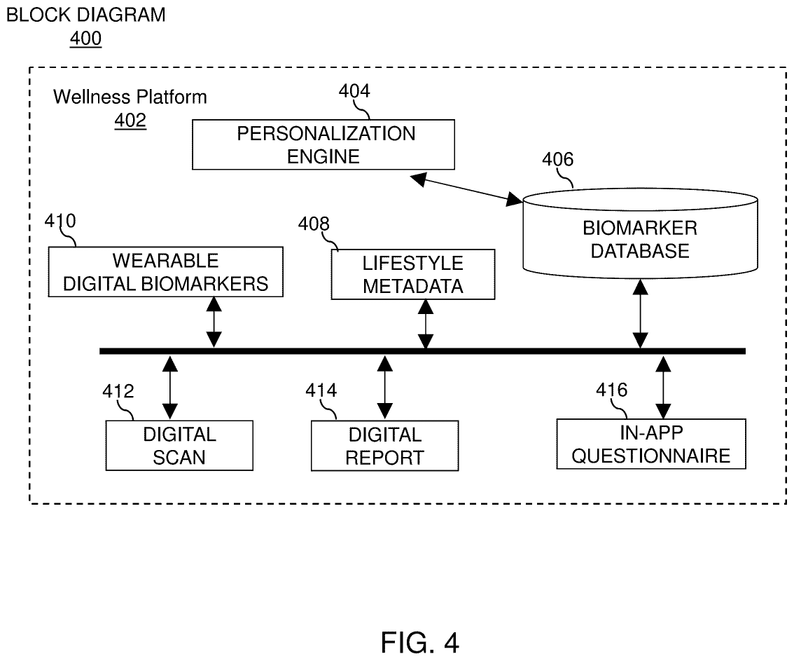

FIG. 4 illustrates a schematic of a wellness platform of a smart community management system according to one embodiment of the present invention. The wellness platform 402 includes a personalization engine 404, a biomarker database 406, lifestyle metadata 408, a wearable digital biomarker component 410, a reporting component 412, a digital scan component 414, and a physiological intake (e.g., questionnaire) component 416. The personalization engine 404 receives biomarker data from the biomarker database 406, lifestyle metadata 408, biomarker data from the wearable digital biomarker component 410, physical data from the digital scan component 412, one or more reports via a remote device and/or third party corresponding to the reporting component 414, and user profile information from the physiological intake component 416. The lifestyle metadata 408 includes information that improves the lifestyle and wellbeing of an individual. For example, and not limitation, the lifestyle metadata includes sleep information such a recommended amount of sleep, an actual amount of sleep, amount of light during sleep, amount of blue light prior to sleep, and other similar information. The wearable digital biomarker component 410 includes at least one remote wearable device designed to monitor and capture biomarker data of a wearer. For example, and not limitation, the wearable digital biomarker component includes a watch, a shirt, a headband, a hat, a wearable patch, and other wearables. The reporting component 414 includes medical reports (e.g., urinalysis, genetic testing).

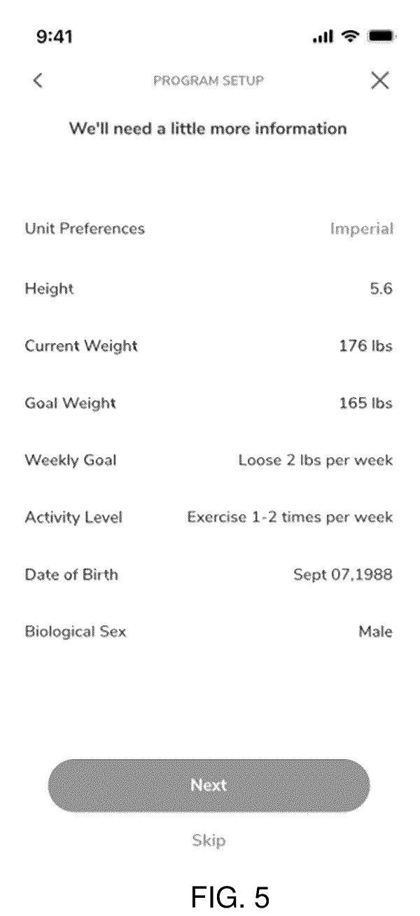

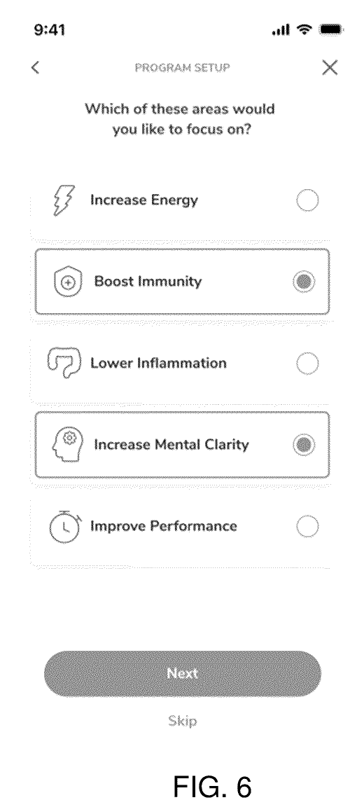

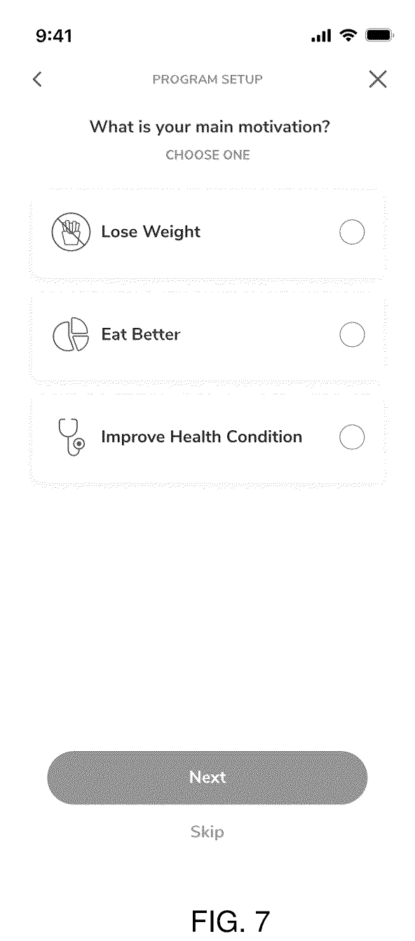

FIG. 5 illustrates a profile dashboard according to one embodiment of the present invention. For example, and not limitation, the present invention includes a software platform designed to receive user physiological data via an interface of a remote device. The user physiological data includes, but is not limited to, a unit preference (e.g., imperial, metric), height, current weight, a goal weight, a weekly goal (e.g., lose 2 pounds per week), activity level, date of birth, sex, and other physiological information. FIG. 6 illustrates a screenshot of a software platform according to one embodiment of the present invention. The software platform is operable to prompt users to select a wellness goal. The wellness goal includes, but is not limited to, increasing energy levels, boosting immunity, lowering inflammation, increasing mental clarity, and improving fitness performance. As shown in FIG. 7, the present invention is further operable to request and/or identify a motivation for a user. For example, and not limitation, a user motivation includes losing weight, improving eating habits, and/or improving overall health condition.

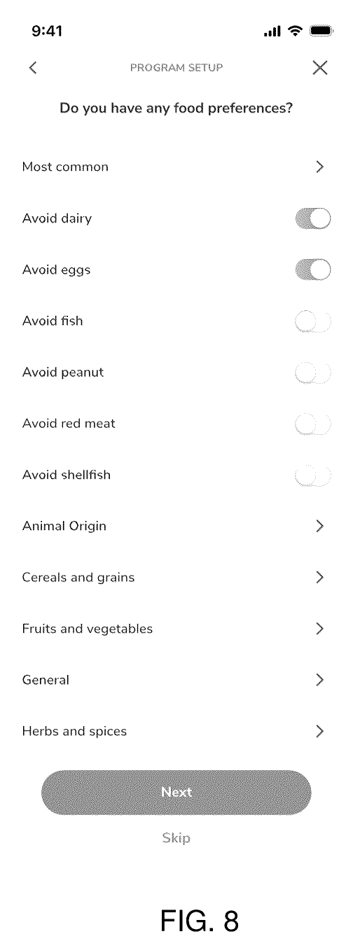

FIG. 8 illustrates a screenshot of a nutrition questionnaire according to one embodiment of the present invention. The software platform is designed to receive user input related to the nutrition preferences and/or allergies of a user. For example, and not limitation, the software platform is operable to receive user input via a touch screen supporting swiping, drag and click, and other similar functionality. The selection of a food preference is made via the touch screen of a remote device. The food preference includes (1) avoid dairy, (2) avoid eggs, (3) avoid fish, (4) avoid peanut, (5) avoid red meat, (6) avoid shellfish, (7) animal original, (8) cereals and grains, fruits and vegetables, and herbs and spices.

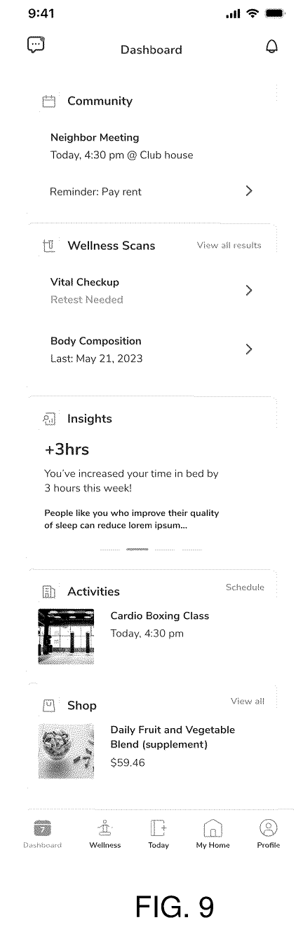

FIG. 9 illustrates a community dashboard of a smart community management system according to one embodiment of the present invention. The community dashboard includes a chat feature, community reminders, wellness information, insights, community activities, and community shops. Advantageously, each section of the community dashboard can be customized to a particular user based on the user's preferences and information. For example, and not limitation, if a user likes swimming, then the software platform is operable to include a swim class under the community activities section.

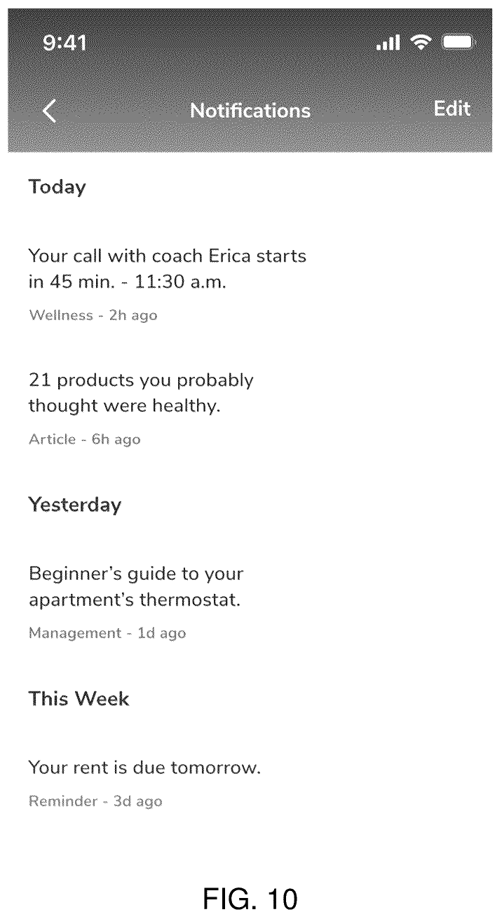

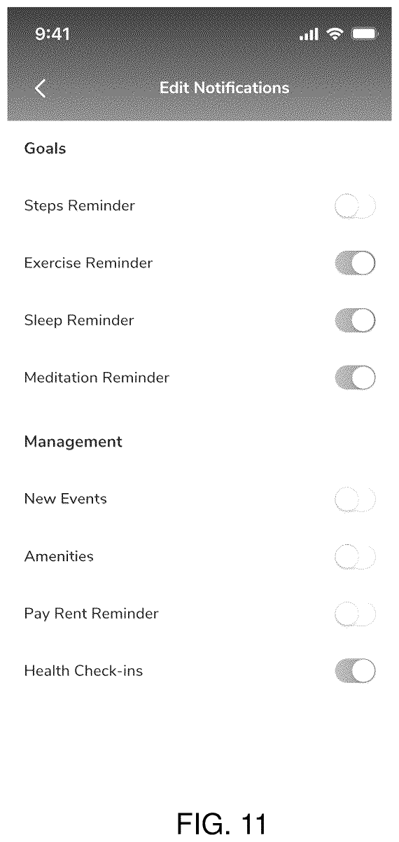

FIG. 10 illustrates a notification dashboard according to one embodiment of the present invention. The notification dashboard, in one embodiment, is separated by time and based on a user's preferences, activities, and physiological information. FIG. 11 illustrates a screenshot of a notification dashboard according to one embodiment of the present invention. The notifications shown on the notification dashboard include, but are not limited to, step reminders, exercise reminders, sleep reminders, meditation reminders, management reminders, events reminders, amenities notifications, rent notifications, and health check-in notifications.

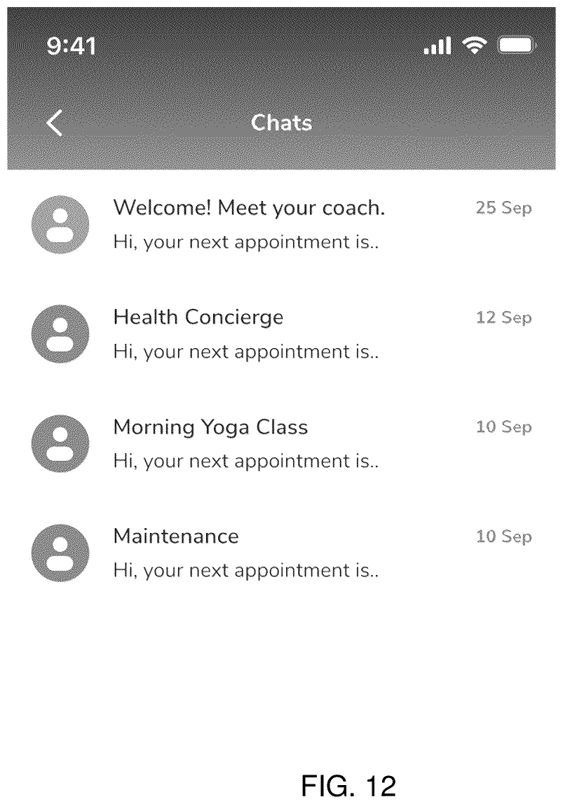

FIG. 12 illustrates a messaging component of a software platform of a smart community management and wellness system according to one embodiment of the present invention. The messaging component is operable to send and maintain communications (e.g., alerts, message, notifications, phone calls) with all aspects of the community of the smart community management and wellness system. For example, and not limitation, the present invention is operable to support electronic communication between neighbors, coaches, doctors, amenities, property management, and other members of a community.

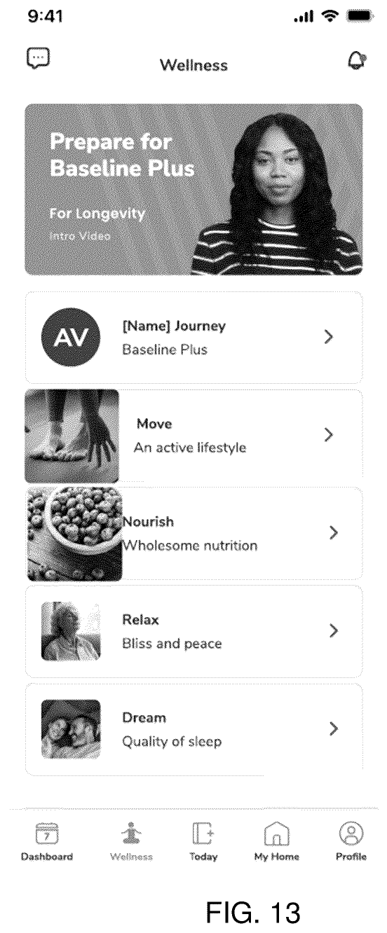

FIG. 13 illustrates a screenshot of a wellness dashboard according to one embodiment of the present invention. The wellness dashboard includes a movement section, a nourishment section, a relax section, and a dream section. The movement section is directed to providing information for the improvement of the fitness and activity of a user. The nourishment section is designed to provide information for improving the nutrition of a user. The relax section is directed toward maintaining emotional and mental health through activities such as mediation. The dream section is directly to monitoring and management of sleep health.

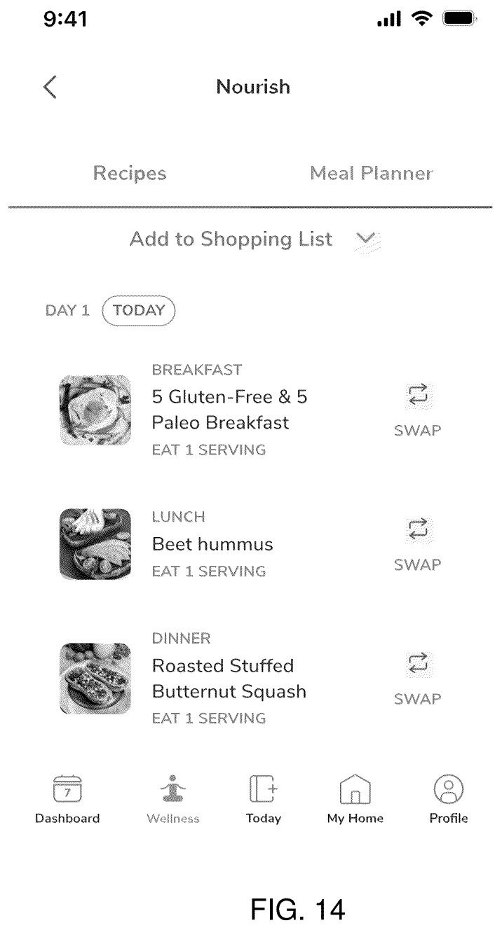

FIG. 14 illustrates an example of the nourishment section of a software platform of a smart community management and wellness system according to one embodiment of the present invention. The nourishment section is designed to provide recipes and meal planning. For example, and not limitation, the meal planner includes meal suggestions and/or recommendations for breakfast, lunch, and/or dinner. Advantageously, the software platform is operable to receive user selection of “add to shopping list” and automatically create a shopping list corresponding to ingredients needed for a recipe, meal suggestion, and/or meal planning Yet another advantage, the present invention is operable to monitor the grocery supplies of a user and to limit the grocery shopping list to items that a user does not have in the user's residence.

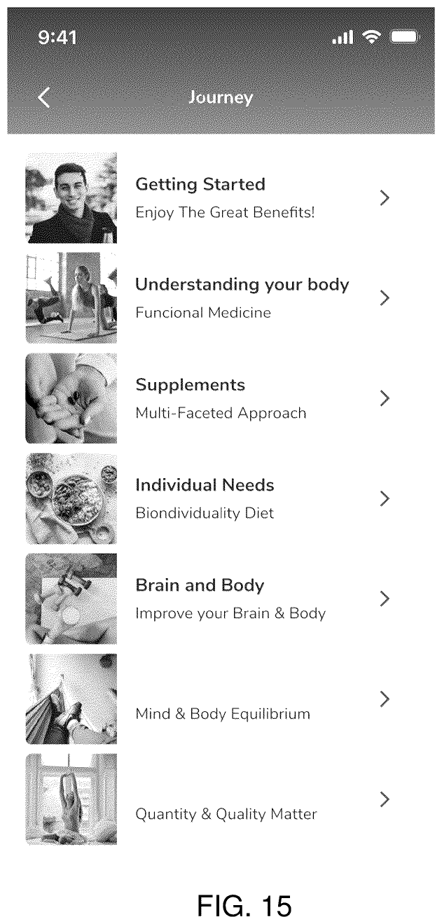

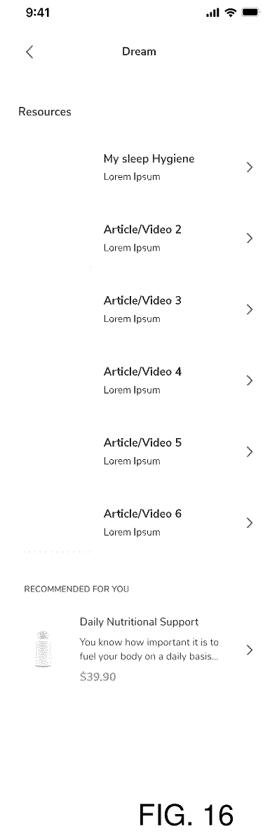

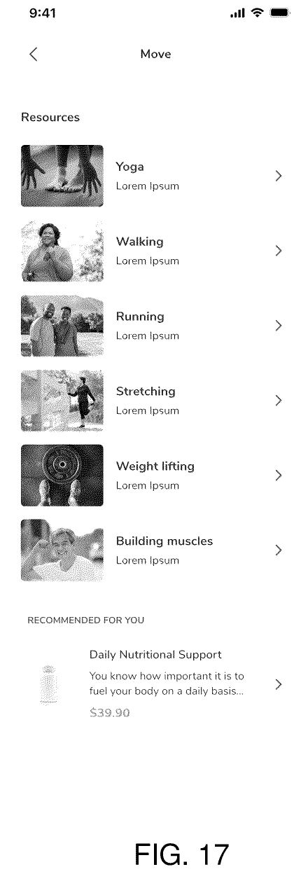

FIG. 15 illustrates a wellness platform of a software platform according to an embodiment of the present invention. The software platform further includes a resources section as shown in FIG. 16. FIG. 17 illustrates an example of a resource section for lifestyle optimization for fitness-related purposes.

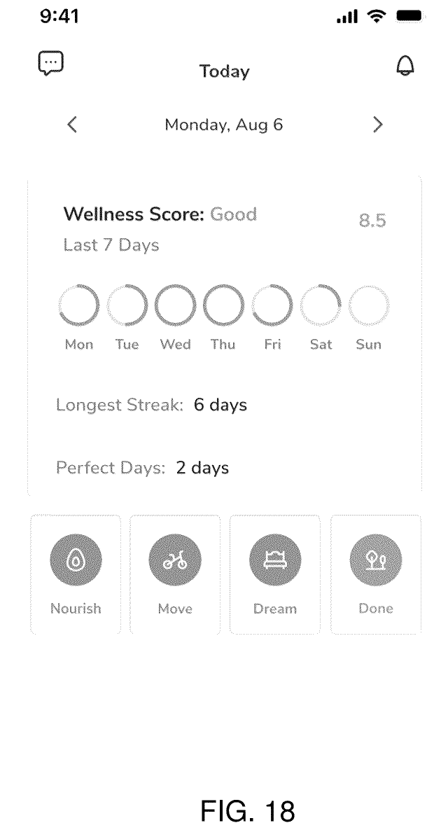

FIG. 18 illustrates a screenshot of a wellness dashboard according to one embodiment of the present invention. As shown in FIG. 18, the software platform is operable to generate a wellness score for a user for a day. The software platform is further operable to track the wellness score over time and to display trends and data over time. The wellness score includes a nourishment component, a movement component, and a sleep component.

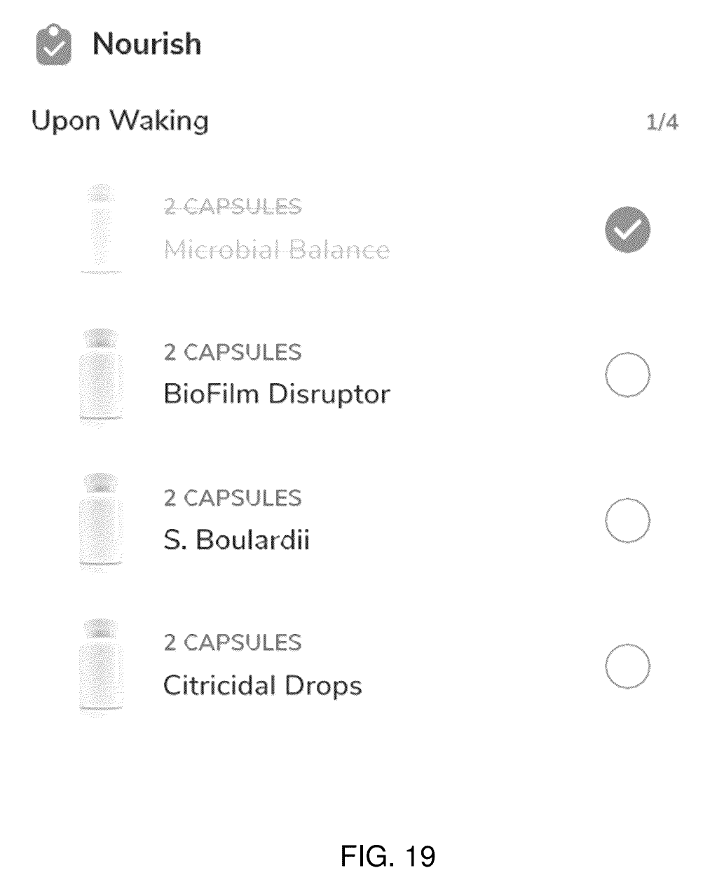

FIG. 19 illustrates a screenshot of a nourishment dashboard according to one embodiment of the present invention. For example, and not limitation, the smart community management and wellness system is operable to provide medication recommendations and to track and notify a user when medication is needed. Advantageously, the software platform is operable to receive user confirmation after the medication is taken.

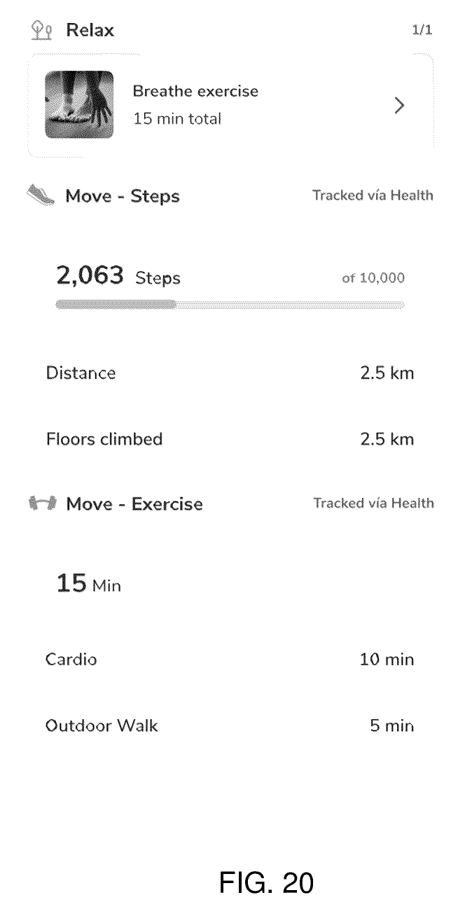

FIG. 20 illustrates a wellness summary corresponding to the movement of a user. The movement summary includes the steps a user took, the amount of a breathing exercise completed, the distance traveled, floors climbed, amount of exercise time, and the type of exercise (e.g., cardio).

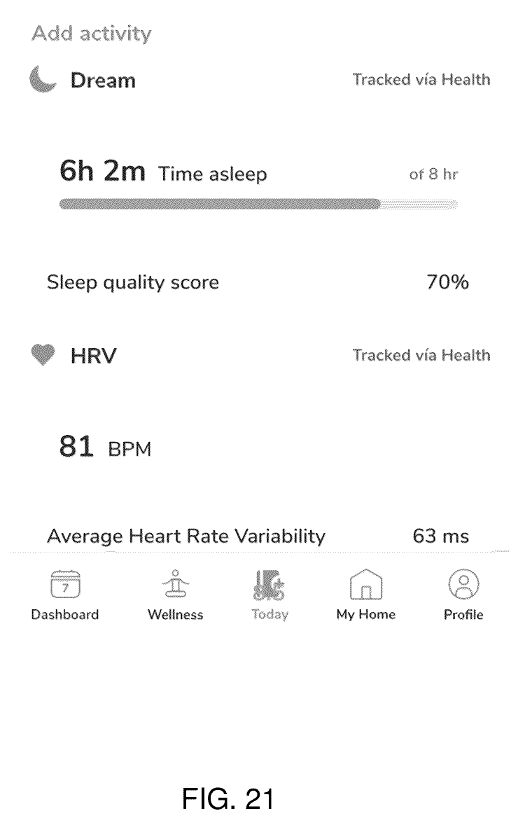

FIG. 21 illustrates a sleep summary and a heart health summary according to one embodiment of the present invention. For example, and not limitation, the sleep summary includes the amount of time slept and the quality of sleep. For further example, and not limitation, the heart summary includes the heart rate of a user and average heart rate variability.

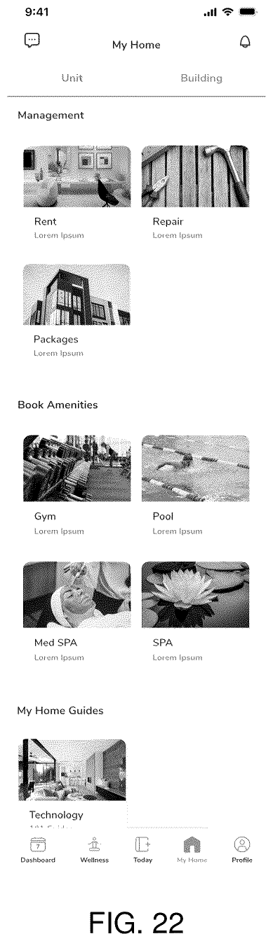

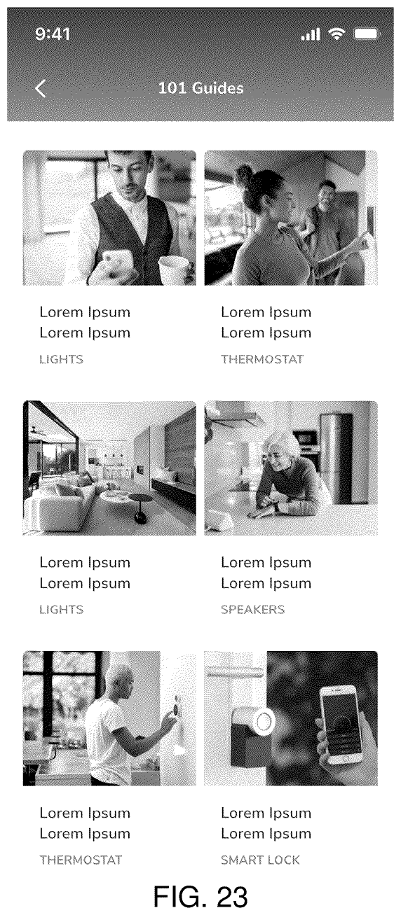

FIG. 22 illustrates a property dashboard according to one embodiment of the present invention. The dashboard includes a unit and/or building tab designed to review data and control of a corresponding unit, building, and/or property. The property dashboard includes a management component, an amenities component, and a guide section. The management section is designed for rent payment, home repayment, and package management. The amenities section enables a user to schedule amenities (e.g., gym, pool, spa) in a community and/or residence. FIG. 23 illustrates a guide section for home management according to one embodiment of the smart community management and wellness system. For example, and not limitation, the guide section includes data corresponding to the management of lights, thermostats, speakers, and smart locks.

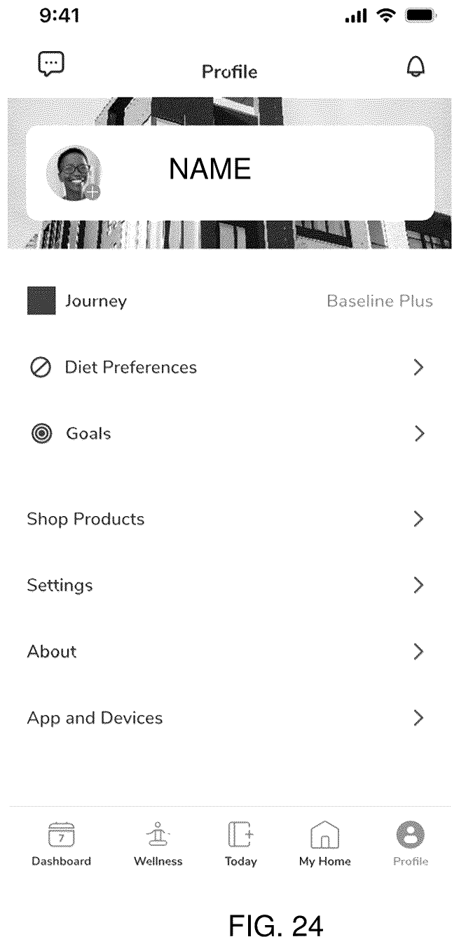

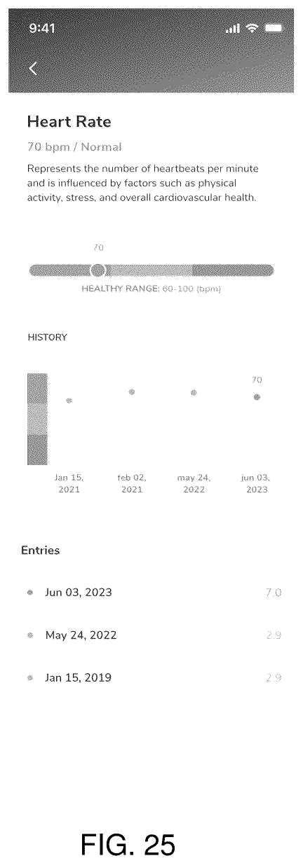

FIG. 24 illustrates a screenshot of a profile dashboard according to one embodiment of the present invention. FIG. 25 illustrates a medical dashboard according to one embodiment of the present invention. For example, and not limitation, the medical dashboard includes real-time heart rate data and historical heart rate data.

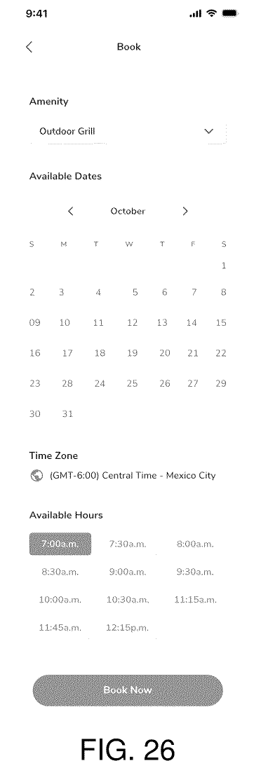

In another embodiment, the smart community management and wellness platform is operable for scheduling various components and features (e.g., amenities). FIG. 26 illustrates a scheduling dashboard according to one embodiment of the present invention. The software platform includes drop-down functionality for a user to select an amenity via a user interface of a remote device. Additionally, the software platform is operable to receive at least one day and time corresponding to an amenity via the user interface of the remote device.

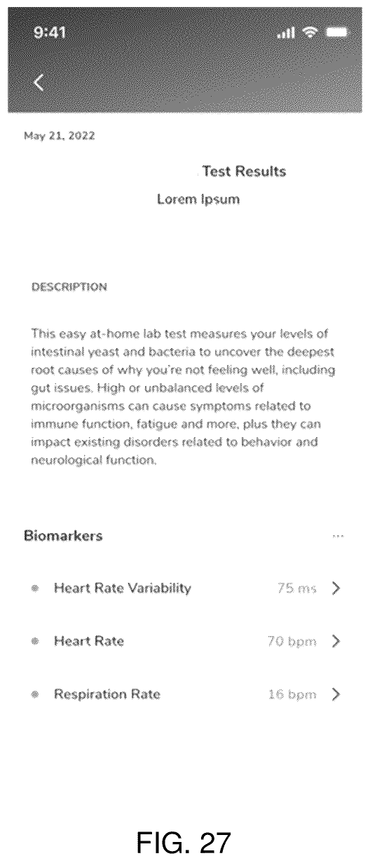

FIG. 27 illustrates a medical dashboard of a smart community management and wellness system according to one embodiment of the present invention. The smart community management and wellness system includes a plurality of medical test and analysis for analyzing the physiological condition of a user. For example, and not limitation, the smart community management and wellness system includes an at-home lab test designed to capture at least one biomarker of a user. In some embodiments, the at-home lab test is operable to generate a result. Alternatively, or additionally, the software platform of the smart community management and wellness system is designed to receive an image of the at-home test and to determine a biomarker based on the received image data. For example, and not limitation, the medical dashboard includes a description of the medical test, and the corresponding biomarkers of the test (e.g., heart rate variability, heart rate, and respiration rate).

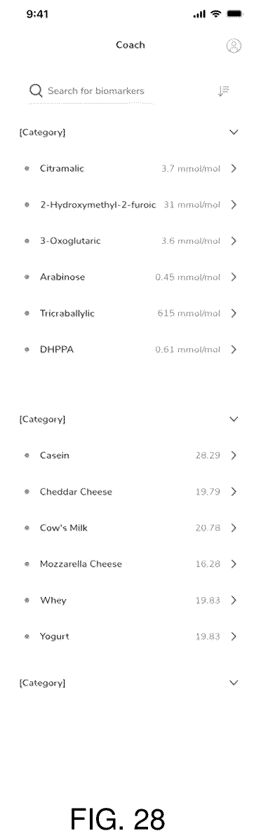

FIG. 28 illustrates a coaching dashboard according to one embodiment of the present invention. The software platform of the smart community management and wellness system is designed to transmit user data (e.g., biomarker data) to a remote device corresponding to a coach. For example, and not limitation, the coaching dashboard includes biomarker data and food data as shown in FIG. 28.

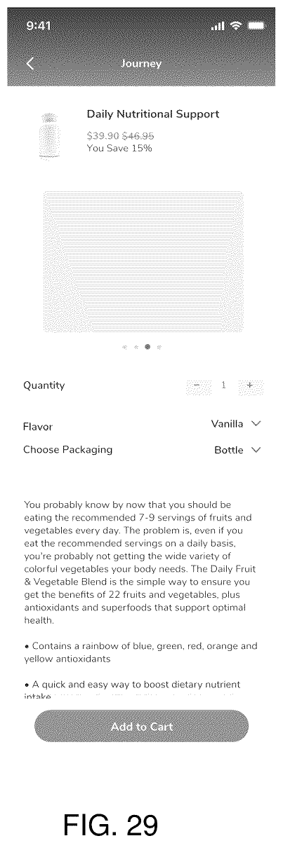

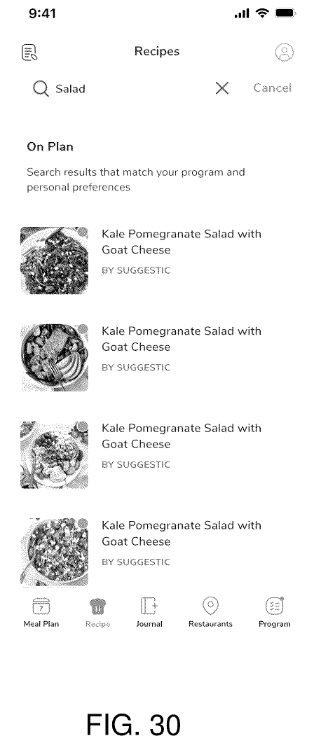

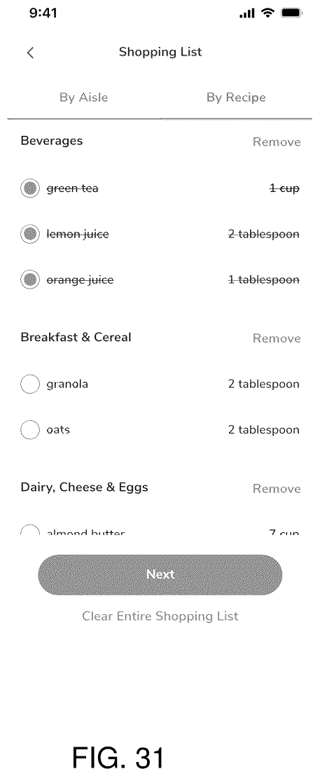

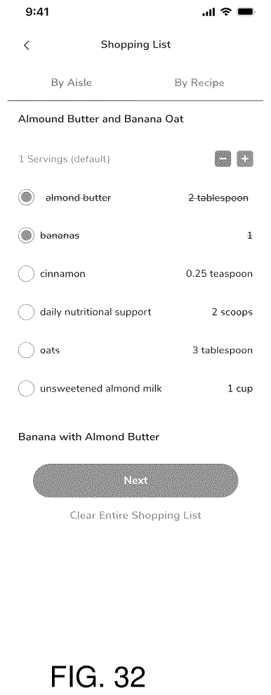

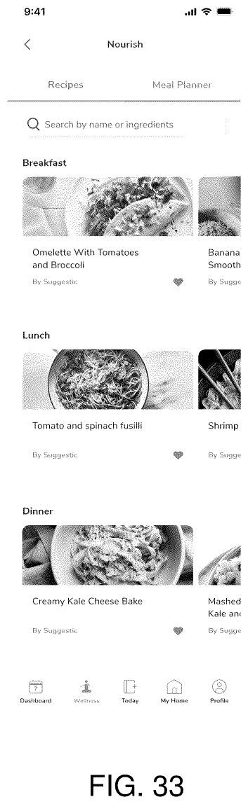

FIG. 29 illustrates a nutritional dashboard of a smart community management and wellness system according to one embodiment of the present invention. The smart community management and wellness system is designed to determine products relevant to the lifestyle optimization of a user. The smart community management and wellness system is further configured to display the determined relevant product to a user via a remote device. Advantageously, the software platform is further operable to provide a corresponding link to add the determined relevant product to an electronic shopping cart (See FIGS. 31 and 32) and/or purchase the product immediately. In some embodiments, the shopping cart is directly linked to a grocery store of the smart community management and wellness system and the software platform is operable to directly submit an order for the relevant product. FIG. 30 illustrates a nutrition dashboard according to one embodiment of the smart community management and wellness system including recipes. Advantageously, the smart community management and wellness systems is designed to automatically add the ingredients for a recipe to a shopping list. An example of a shopping list according to an embodiment of the present invention can be found in FIGS. 31, 32, and 33.

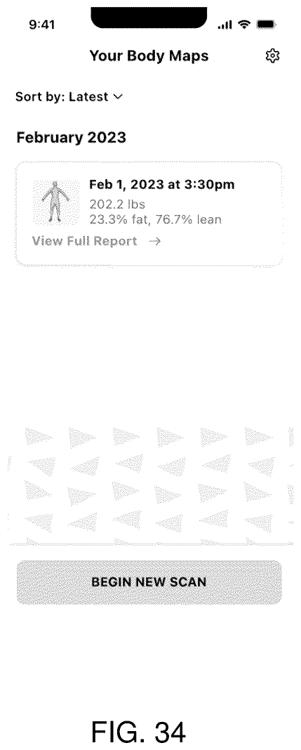

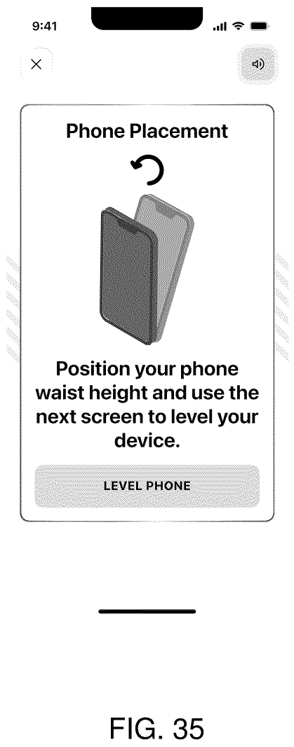

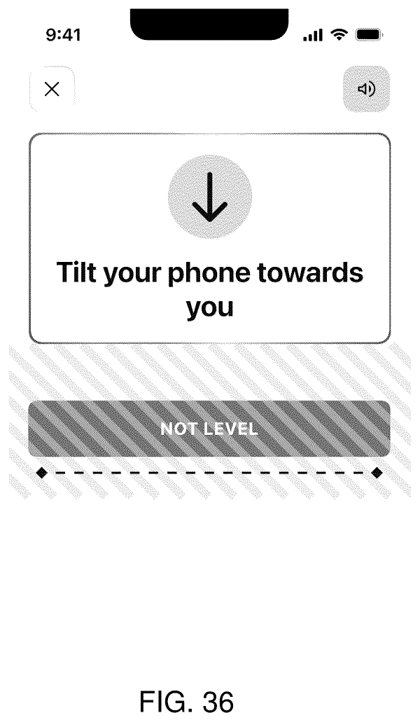

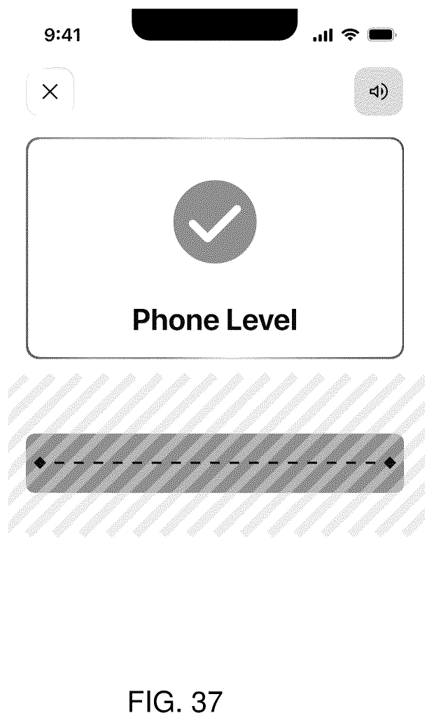

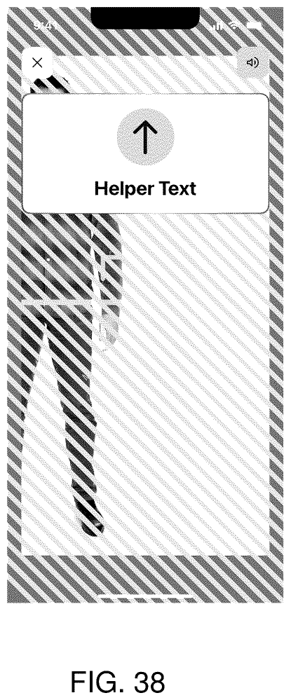

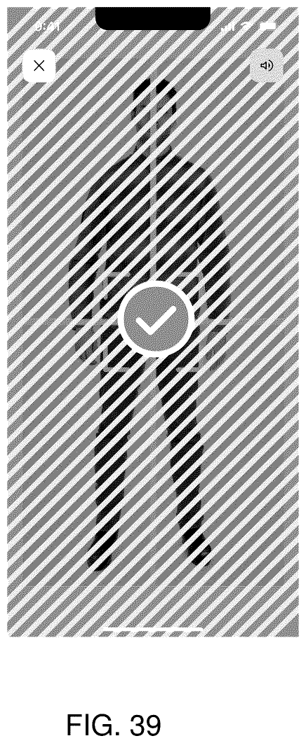

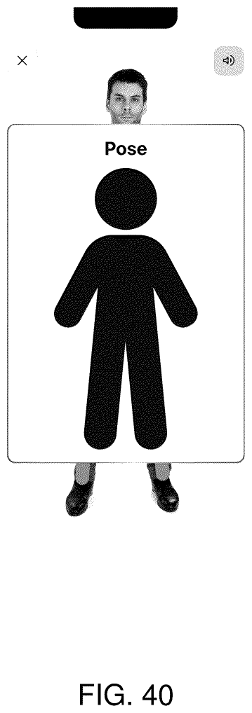

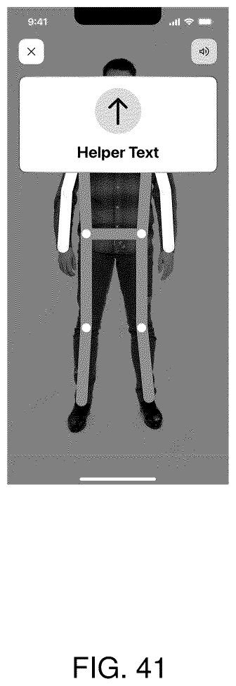

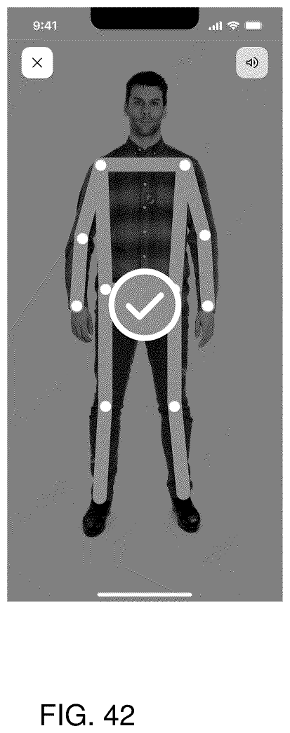

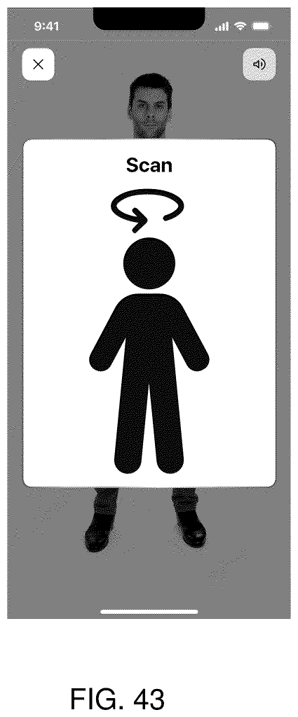

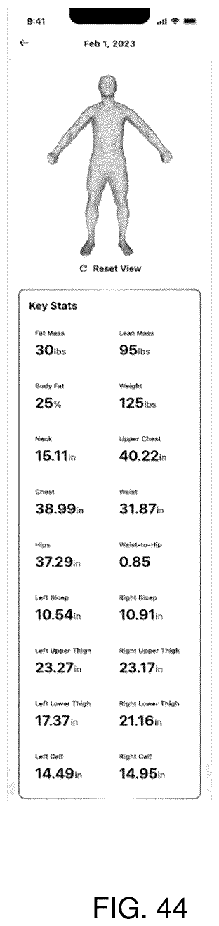

As shown in FIG. 34-44, in some embodiments, the smart community management and wellness system is operable to capture and model a body map of an individual using a remote device. The software platform is designed to provide instructions (as shown in FIGS. 35-43) on using a remote device (e.g., a cellphone) to capture an image of a user and the corresponding movements a user must perform. Advantageously, using the sensors from the remote device, the software platform is operable to determine whether the user and/or remote device is properly positioned to capture the body map of a user. After the body map scan has been performed, the smart community system is operable to determine a user's fat mass, lean mass, body fat, weight, neck size, upper chest size, chest size, waist size, hip size, waist to hip ratio, bicep size, thigh size, and calf size.

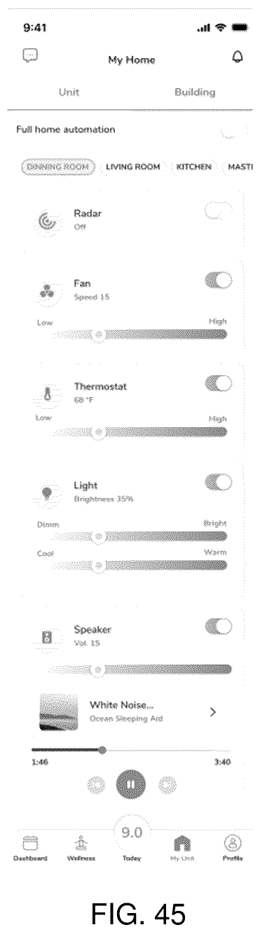

FIG. 45 illustrates a smart home dashboard of a software platform of a smart community management and wellness system according to one embodiment of the present invention. The smart home dashboard is displayable and controllable via a remote device (e.g., mobile device, computer, laptop). The smart home dashboard displays a plurality of appliances and corresponding settings. The smart home dashboard includes any appliance, electronic device, electric device, IoT device, and other devices mentioned throughout this description. For example, and not limitation, the plurality of appliances includes, but is not limited to, a sleep radar, a fan, a thermostat, a light, and a speaker. The smart home dashboard includes a power control feature (e.g., a slider on the electronic user interface) that enables a user to power an appliance on or off. For example, and not limitation, via a user interface of a remote device, the smart home dashboard is operable to receive a selection of power on (slide a slider to a first direction) or a selection of power off (slide to a second direction). For further example, and not limitation, the appliance includes a fan, and a speed setting is controllable via the user interface and/or automatically via the software platform. The speed setting includes but is not limited to a speed selection (low, medium, high), a slidable scale, and/or a speed number received via the user interface. In some embodiments, the appliance includes a thermostat. The temperature setting of the thermostat is displayed via the smart home dashboard. The temperature setting includes but is not limited to a temperature selection (cool, hot), a slidable scale, and/or an desired temperature received via the user interface (e.g., 30 degrees Celsius). In some embodiments, the appliance includes a light appliance with a brightness setting and a color setting. For example, and not limitation, the brightness temperature setting a brightness selection (dim, bright), and/or a slidable scale for changing the brightness of a light. In some embodiments, the speaker appliance includes a volume setting and a corresponding audio file. For example, and not limitation, the volume setting includes a volume level. The audio file includes music, white noise, and similar audio files. The audio file may include a corresponding wellness recommendation (e.g., white noise recommendation).

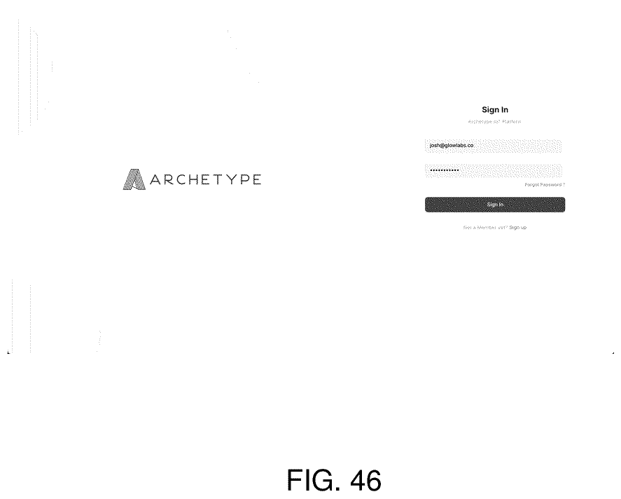

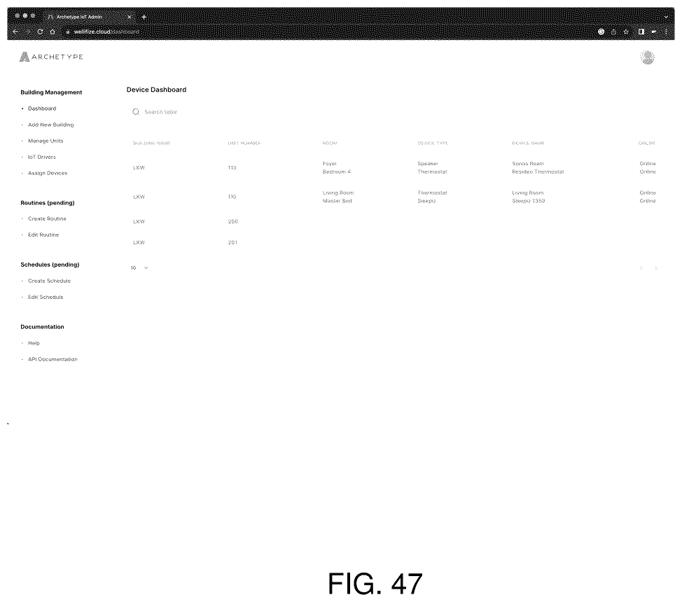

FIG. 46 illustrates a login user interface of a software platform of a smart community management and wellness system according to one embodiment of the present invention. The login user interface is operable to receive user account information via a user interface of a remote device. FIG. 47 illustrates a smart device dashboard of a software platform of a smart community management and wellness system according to one embodiment of the present invention. The device dashboard includes a location (e.g. building, unit, and/or room) of a device, device type, device name, and the connectivity status (e.g., offline, online). The software platform further includes a building management dashboard that enables adding new buildings and other properties, managing a plurality of properties, IoT drivers, and assigning devices to users, properties, and/or remote devices. For further example, yet another advantage of the smart community management and wellness system is manually and/or automatically creating routines and schedules for one or more devices in network communication. For example, and not limitation, the software platform is operable to generate a sleep routine that includes lowering the temperature in a selected unit during a targeted time (e.g., 10 p.m. to 6 a.m.) to improve a user's sleep.

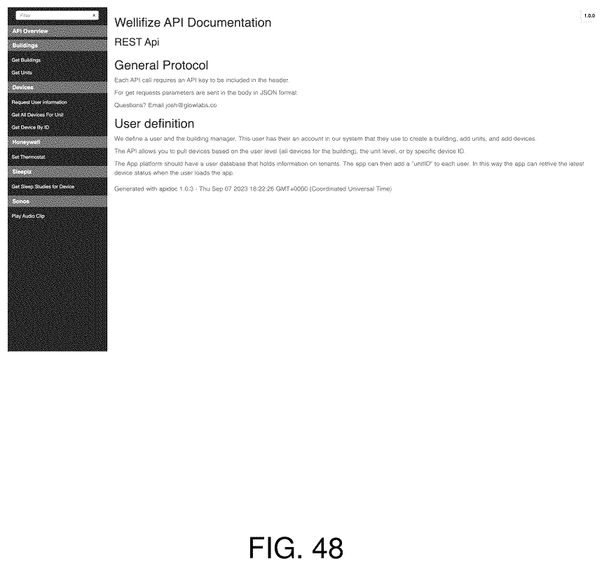

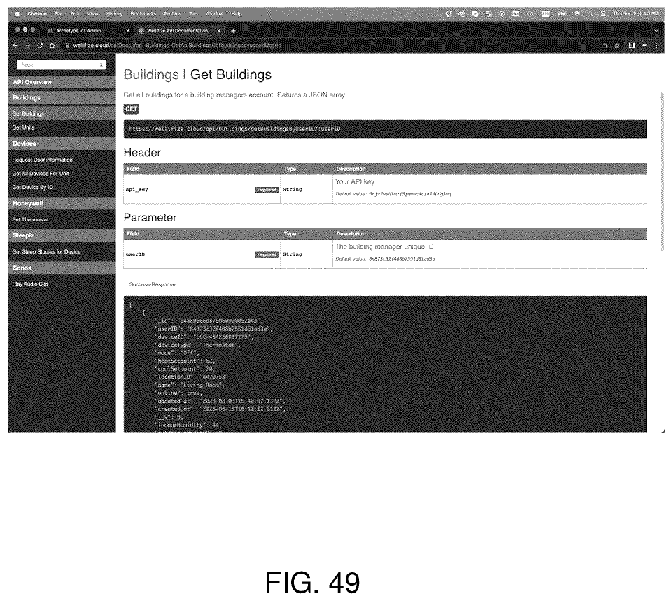

FIG. 48 illustrates wellness application programming interface documentation of a software platform of a smart community management and wellness system according to one embodiment of the present invention. FIG. 49 illustrates building application programming interface documentation of a software platform of a smart community management and wellness system according to one embodiment of the present invention. The smart community management and wellness system is operable to define a user corresponding to a remote device and/or a property (e.g., apartment) and a corresponding building manager. The software platform is operable to receive user selection via a remote device and/or automatically assign a building, units, and/or devices to at least one user. For example, and not limitation, the assigned devices include all devices for each user of a property, for a unit, and/or a specific device. Additional user information for each apartment in a building is included.

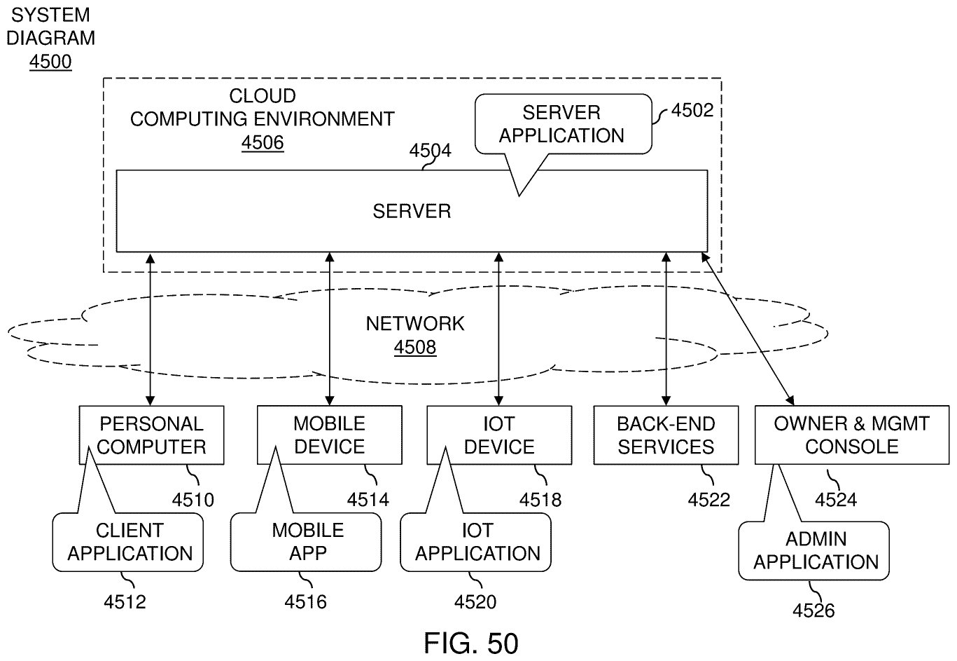

FIG. 50 depicts a system diagram 4500 illustrating a client/server architecture in accordance with embodiments of the present disclosure. The server application 4502 is configured to provide a video application and mobile application for a smart multi-resident community management system. A server application 4502 is hosted on a remote server 4504 within a cloud computing environment 4506. The server application 4502 is provided on a non-transitory computer-readable medium including a plurality of machine-readable instructions, which when executed by one or more processors of the server 4504, are adapted to cause the server 4504 to generate the video platform and mobile application.

The server application 4502 is configured to communicate over a network 4508. In a preferred embodiment, the network 4508 is the Internet. In other embodiments, the network 4508 may be restricted to a private local area network (LAN) and/or private wide area network (WAN). The network 4508 provides connectivity with a plurality of client devices including a personal computer 4510 hosting a client application 4512, a mobile device 4514 hosting a mobile app 4516. The network 4508 also provides connectivity for an Internet-Of-Things (IoT) device 4518 hosting an IoT application 4520, to back-end services 4522, and to an owner and management console including an administration software application. Advantageously, the back-end services are operable to communicate with third-party application programming interfaces (APIs) to either provide or receive data that can be used by the system to provide recommendations. Third-party applications provide algorithms for analysis of data. The back-end services may provide data gathered within the smart community management and wellness systems through the third-party APIs and receives results from the algorithms provided back to the back-end services to provide further recommendations or take further actions within the community management system. The owner and management console 4524 is designed to control and monitor the various features and components (e.g., appliances, amenities) of a community discussed throughout this application

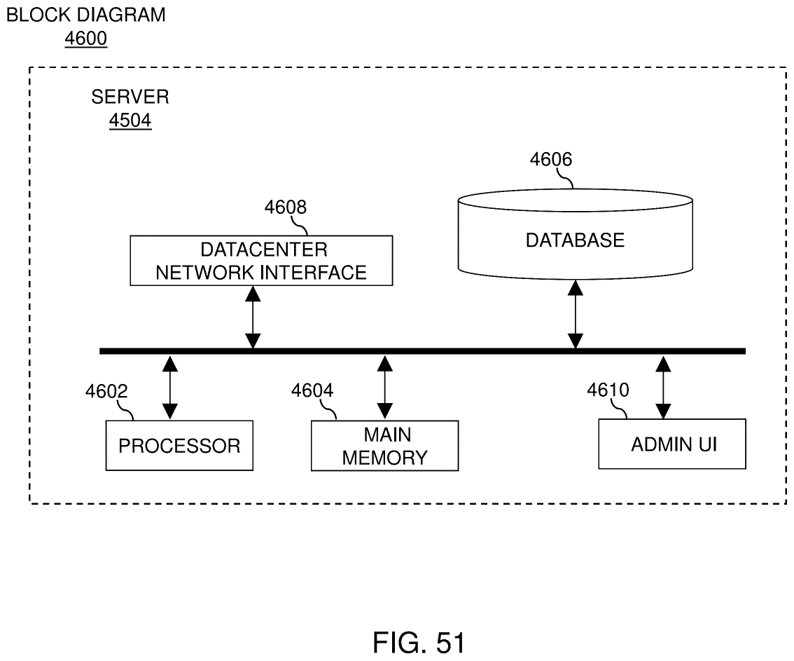

FIG. 51 depicts a block diagram 4600 of the server 4504 of FIG. 50 for hosting at least a portion of the server application 4502 of FIG. 50 in accordance with embodiments of the present disclosure. The server 4504 may be any of the hardware servers referenced in this disclosure. The server 4504 may include at least one of a processor 4602, a main memory 4604, a database 4606, a datacenter network interface 4608, and an administration user interface (UI) 4610. The server 4504 may be configured to host one or more virtualized servers. For example, the virtual server may be an Ubuntu® server or the like. The server 4504 may also be configured to host a virtual container. For example, the virtual server may be the DOCKER® virtual server or the like. In some embodiments, the virtual server and or virtual container may be distributed over a plurality of hardware servers using hypervisor technology.

The processor 4602 may be a multi-core server class processor suitable for hardware virtualization. The processor 4602 may support at least a 64-bit architecture and a single instruction multiple data (SIMD) instruction set. The memory 4604 may include a combination of volatile memory (e.g., random access memory) and non-volatile memory (e.g., flash memory). The database 4606 may include one or more hard drives.

The datacenter network interface 4608 may provide one or more high-speed communication ports to the data center switches, routers, and/or network storage appliances. The datacenter network interface may include high-speed optical Ethernet, InfiniBand (IB), Internet Small Computer System Interface iSCSI, and/or Fibre Channel interfaces. The administration UI may support local and/or remote configuration of the server by a data center administrator.

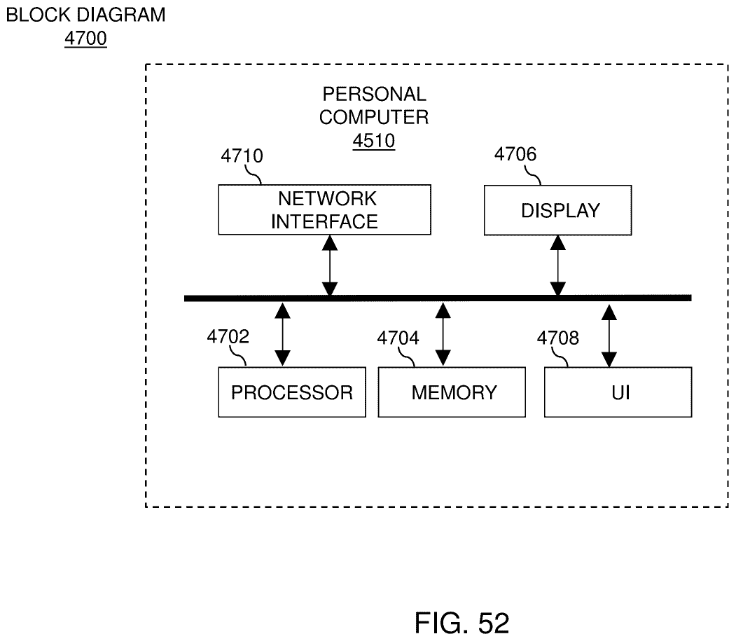

FIG. 52 depicts a block diagram 4700 of the personal computer 4510 of FIG. 50 in accordance with embodiments of the present disclosure. The personal computer 4510 may be any of the devices referenced in this disclosure. The personal computer 4510 may include at least a processor 4702, a memory 4704, a display 4706, a user interface (UI) 4708, and a network interface 4710. The personal computer 4510 may include an operating system to run a web browser and/or the client application 4512 shown in FIG. 50. The operating system (OS) may be a Windows® OS, a Macintosh® OS, or a Linux® OS. The memory 4704 may include a combination of volatile memory (e.g., random access memory) and non-volatile memory (e.g., solid state drive and/or hard drives).

The network interface 4710 may be a wired Ethernet interface or a Wi-Fi interface. The personal computer 4510 may be configured to access remote memory (e.g., network storage and/or cloud storage) via the network interface 4710. The UI 4708 may include a keyboard, and a pointing device (e.g., mouse). The display 4706 may be an external display (e.g., computer monitor) or internal display (e.g., laptop). In some embodiments, the personal computer 4510 may be a smart TV. In other embodiments, the display 4706 may include a holographic projector.

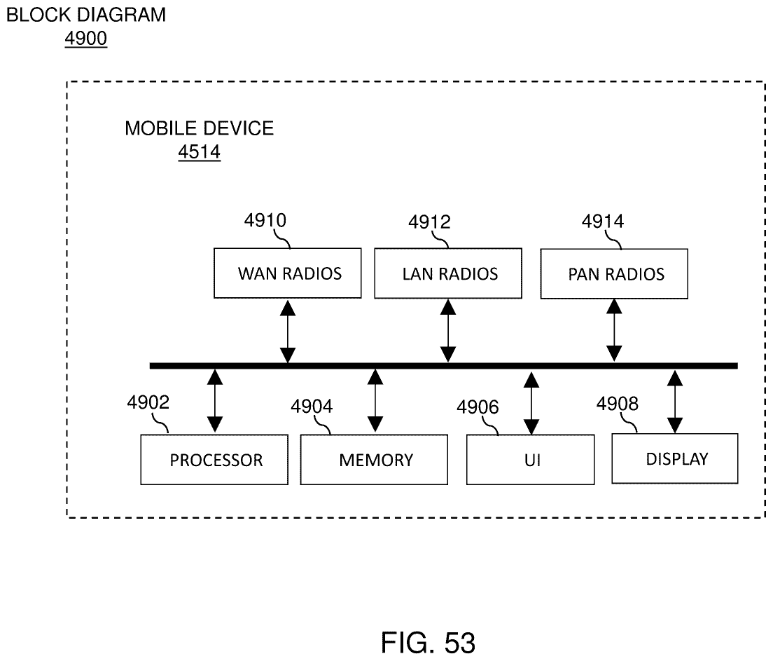

FIG. 53 depicts a block diagram 4800 of the mobile device 4514 of FIG. 50 in accordance with embodiments of the present disclosure. The mobile device 4514 may be any of the remote devices referenced in this disclosure. The mobile device 4514 may include an operating system to run a web browser and/or the mobile app 4516 shown in FIG. 50. The mobile device 4514 may include at least a processor 4802, a memory 4804, a UI 4806, a display 4808, WAN radios 4810, LAN radios 4812, and personal area network (PAN) radios 4814. In some embodiments the mobile device 4514 may be an iPhone® or an iPad®, using iOS® as an OS. In other embodiments, the mobile device 4514 may be a mobile terminal including Android® OS, BlackBerry® OS, Chrome® OS, Windows Phone® OS, or the like.

In some embodiments, the processor 4802 may be a mobile processor such as the Qualcomm® Snapdragon™ mobile processor. The memory 4804 may include a combination of volatile memory (e.g., random access memory) and non-volatile memory (e.g., flash memory). The memory 4804 may be partially integrated with the processor 4802. The UI 4806 and display 4808 may be integrated such as a touchpad display. The WAN radios 4810 may include 2G, 3G, 4G, and/or 5G technologies. The LAN radios 4812 may include Wi-Fi technologies such as 802.11a, 802.11b/g/n, and/or 802.11ac circuitry. The PAN radios 4814 may include Bluetooth® technologies.

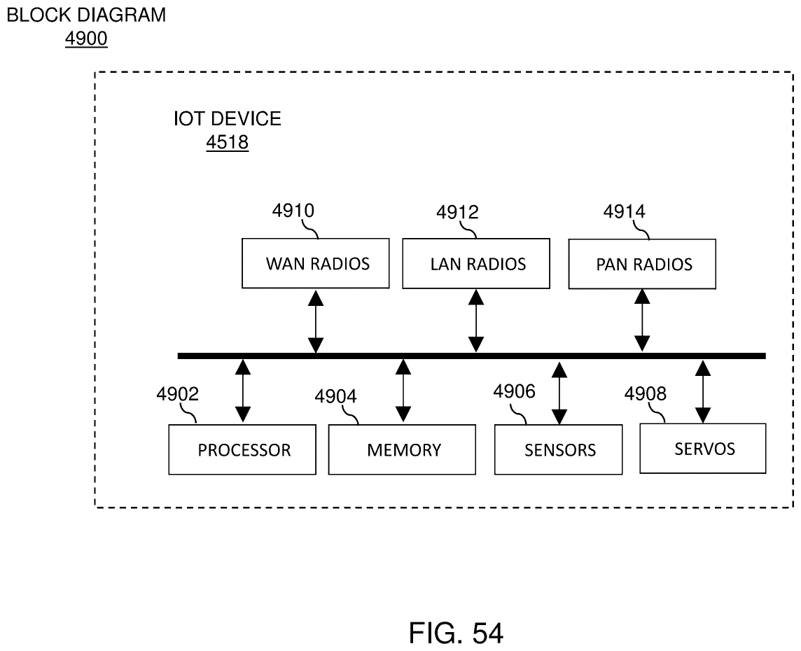

FIG. 54 depicts a block diagram 4900 of the IoT device 4518 of FIG. 50 in accordance with embodiments of the present disclosure. The IoT device 4518 may be any of the remote devices referenced in this disclosure. The IoT device 4518 includes a processor 4902, a memory 4904, sensors 4906, servos 4908, WAN radios 4910, LAN radios 4912, and PAN radios 4914. The processor 4902, a memory 4904, WAN radios 4910, LAN radios 4912, and PAN radios 4914 may be of similar design to the processor 4802, a memory 4804, WAN radios 4810, LAN radios 4812, and PAN radios 4814 of the mobile device 4514 of FIG. 53. The sensors 4906 and servos 4908 may include any applicable components related to IoT devices such as a monitoring device, an autonomous vehicle, a home assistant, a smart appliance, a medical device, a virtual reality device, an augmented reality device, or the like.

Any combination of one or more computer-readable medium(s) may be utilized. The computer-readable medium may be a computer readable signal medium or a computer-readable storage medium (including, but not limited to, non-transitory computer-readable storage media). A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer readable storage medium and that can communicate, propagate, or transport a program for use by or in connection with an instruction execution system, apparatus, or device.

In one embodiment, the present invention includes a cloud-based network for distributed communication via a wireless communication antenna and processing by at least one mobile communication computing device. In another embodiment of the invention, the system is a virtualized computing system capable of executing any or all aspects of software and/or application components presented herein on computing devices. In certain aspects, the computer system may be implemented using hardware or a combination of software and hardware, either in a dedicated computing device, or integrated into another entity, or distributed across multiple entities or computing devices.

By way of example, and not limitation, the computing devices are intended to represent various forms of digital computers and mobile devices, such as a server, blade server, mainframe, mobile phone, personal digital assistant (PDA), smartphone, desktop computer, netbook computer, tablet computer, workstation, laptop, and other similar computing devices. The components shown here, their connections and relationships, and their functions, are meant to be exemplary only, and are not meant to limit implementations of the invention described and/or claimed in this document.

In one embodiment, the computing device includes components such as a processor, a system memory having a random-access memory (RAM) and a read-only memory (ROM), and a system bus that couples the memory to the processor. In another embodiment, the computing device may additionally include components such as a storage device for storing the operating system and one or more application programs, a network interface unit, and/or an input/output controller. Each of the components may be coupled to each other through at least one bus. The input/output controller may receive and process input from, or provide output to, a number of other devices, including, but not limited to, alphanumeric input devices, mice, electronic styluses, display units, touch screens, signal generation devices (e.g., speakers), or printers.

By way of example, and not limitation, the processor may be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another embodiment, multiple processors and/or multiple buses may be used, as appropriate, along with multiple memories of multiple types (e.g., a combination of a DSP and a microprocessor, a plurality of microprocessors, one or more microprocessors in conjunction with a DSP core).

Also, multiple computing devices may be connected, with each device providing portions of the necessary operations (e.g., a server bank, a group of blade servers, or a multi-processor system). Alternatively, some steps or methods may be performed by circuitry that is specific to a given function. According to various embodiments, the computer system may operate in a networked environment using logical connections to local and/or remote computing devices through a network. A computing device may connect to a network through a network interface unit connected to a bus. Computing devices may communicate communication media through wired networks, direct-wired connections or wirelessly, such as acoustic, RF, or infrared, through an antenna in communication with the network antenna and the network interface unit, which may include digital signal processing circuitry when necessary. The network interface unit may provide for communications under various modes or protocols.

In one or more exemplary aspects, the instructions may be implemented in hardware, software, firmware, or any combinations thereof. A computer readable medium may provide volatile or non-volatile storage for one or more sets of instructions, such as operating systems, data structures, program modules, applications, or other data embodying any one or more of the methodologies or functions described herein. The computer readable medium may include the memory, the processor, and/or the storage media and may be a single medium or multiple media (e.g., a centralized or distributed computer system) that store the one or more sets of instructions. Non-transitory computer readable media includes all computer readable media, with the sole exception being a transitory, propagating signal per se. The instructions may further be transmitted or received over the network via the network interface unit as communication media, which may include a modulated data signal such as a carrier wave or other transport mechanism and includes any delivery media. The term “modulated data signal” means a signal that has one or more of its characteristics changed or set in a manner as to encode information in the signal.

Storage devices and memory include, but are not limited to, volatile and non-volatile media such as cache, RAM, ROM, EPROM, EEPROM, FLASH memory, or other solid state memory technology; discs (e.g., digital versatile discs (DVD), HD-DVD, BLU-RAY, compact disc (CD), or CD-ROM) or other optical storage; magnetic cassettes, magnetic tape, magnetic disk storage, floppy disks, or other magnetic storage devices; or any other medium that can be used to store the computer readable instructions and which can be accessed by the computer system.

The various illustrative logical blocks, modules, elements, circuits, and algorithms described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present invention.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A smart community management and wellness system comprising:
at least one remote device including a user interface;
at least one image sensor configured to capture an image; and
at least one remote server including a software platform including a plurality of dashboards, an analytics engine, and at least one database;
wherein the at least one remote device, the at least one image sensor, and the at least one remote server are in network communication;
wherein the at least one image sensor is operable to capture image data corresponding to at least one user, wherein the user image data is transmitted to the at least one remote device and the at least one remote server;
wherein, after receiving the user image data, the at least one remote device is operable to display the user image data via the user interface;
wherein, after receiving the user image data, the analytics engine of the at least one remote server is designed to analyze the user image data to determine at least one physiological condition of the user;
wherein, based on the at least one determined physiological condition of the user, the at least one remote server is operable to transmit the at least one determined physiological condition to the at least one remote device, wherein the at least one remote device is operable to display the at least one determined physiological condition; and
wherein the at least one remote server is further operable to provide at least one wellness recommendation based on the at least one determined physiological condition, wherein the at least one wellness recommendation includes a sleep recommendation, a nutrition recommendation, a mental health recommendation, and/or a fitness recommendation.

2. The system of claim 1, wherein the smart community management and wellness system further includes a plurality of controllable electronic devices and a plurality of controllable electric devices, wherein the plurality of controllable electronic devices and the plurality of controllable electric devices are in network communication with the at least one remote server and the at least one remote device, wherein the at least one remote server is operable to transmit an activation command to at least one controllable electronic device of the plurality of controllable electronic devices and/or at least one controllable electric device of the plurality of controllable electric devices based on the at least one determined physiological condition and the at least one wellness recommendation.

3. The system of claim 2, wherein the at least one determined physiological condition includes at least one biomarker, wherein the at least one database further includes historical user data and controllable electronic device data, wherein the historical user data includes biomarker data corresponding to at least one user, wherein the controllable electronic device data includes a power status and at least one setting corresponding to at least one controllable electronic device of the plurality of controllable electronic devices, wherein the analytics engine is further operable to generate at least one controllable electronic device recommendation based on the historical user data and the controllable electronic device data, wherein the at least one controllable electronic device recommendation includes a change of the power status and/or of the at least one setting.

4. The system of claim 1, wherein the at least one determined physiological condition includes at least one biomarker.

5. The system of claim 1, wherein the at least one image sensor is operable to receive at least one command from the at least one remote device, wherein the at least one command includes a request for user image data, wherein, in response to the at least one command, the at least one image sensor is activated.

6. The system of claim 1, further comprising at least one wearable device, wherein the at least one wearable device includes a positioning component, wherein the positioning component is operable to track a geographical position of the at least one user, wherein the at least one remote server is operable to modify the at least one wellness recommendation based on user position.

7. The system of claim 6, wherein the at least one database further includes property data and location data for a predetermined geographic area, wherein the property data includes building data and amenities data, wherein the amenities data includes a plurality of amenities in the predetermined geographic area, wherein the at least one wellness recommendation further includes at least one amenity of the plurality of amenities in the predetermined geographic area.

8. A smart community management and wellness system comprising:
at least one remote device including a user interface;
at least one controllable electronic device;
at least one image sensor configured to capture an image; and
at least one remote server including a software platform including a plurality of dashboards, an analytics engine, and at least one database, wherein the at least one database includes user data, property data, electronic device data, and health data;
wherein the at least one remote device, the at least one image sensor, the at least one controllable electronic device, and the at least one remote server are in network communication;
wherein the at least one image sensor is operable to capture image data corresponding to at least one user, wherein the user image data is transmitted to the at least one remote device and the at least one remote server;
wherein, after receiving the user image data, the at least one remote device is operable to display the user image data via the user interface;
wherein, after receiving the user image data, the analytics engine of the at least one remote server is designed to analyze the user image data to determine at least one physiological condition of the user;
wherein based on the at least one determined physiological condition of the user, the at least one remote server is operable to transmit the at least one determined physiological condition to the at least one remote device, wherein the at least one remote device is operable to display the at least one determined physiological condition;
wherein the at least one remote server is further operable to provide at least one wellness recommendation based on the at least one determined physiological condition, wherein the at least one wellness recommendation includes a sleep recommendation, a nutrition recommendation, a mental health recommendation, and/or a fitness recommendation; and
wherein the at least one remote server is further operable to transmit an activation command to the at least one controllable electronic device based on the at least one determined physiological condition.

9. The system of claim 8, wherein the sleep recommendation includes an amount of sleep for the at least one user, wherein the nutrition recommendation includes a food or a liquid for consumption by the at least one user, and wherein the fitness recommendation includes at least one exercise.

10. The system of claim 8, wherein the analytics engine is operable to determine at least one trend based on the at least one determined physiological condition, wherein, based on the at least one trend, the analytics engine is operable to generate at least one habit recommendation, wherein the at least one habit recommendation includes at least one action corresponding to the at least one user for creating a habit.

11. The system of claim 8, wherein the user data includes medical information corresponding to the at least one user, wherein the property data includes at least one residence of the at least one user, wherein the at least one controllable electronic device is located on the residence of the at least one user, wherein the at least one wellness recommendation is based on the user medical information and the at least one determined physiological condition, wherein the at least one wellness recommendation includes at least one setting corresponding to the at least one controllable electronic device.

12. The system of claim 8, further comprising at least one wearable device in network communication with the at least one remote device and the at least one remote server, wherein the at least one wearable device includes at least one sensor and a positioning component, wherein the at least one sensor is operable to monitor a physiological condition of the at least one user, wherein the positioning component is operable to track a geographical position of the at least one user, wherein the at least one remote server is operable to modify the at least one wellness recommendation based on the user sensor data and the user position.

13. The system of claim 12, wherein the at least one database further includes property data and location data for a predetermined geographic area, wherein the property data includes building data and amenities data, wherein the amenities data includes a plurality of amenities in the predetermined geographic area, wherein the at least one wellness recommendation further includes at least one amenity of the plurality of amenities in the predetermined geographic area.

14. A smart community management and wellness system comprising:
a plurality of remote devices, wherein each remote device of the plurality of remote devices includes a user interface;
a plurality of controllable electronic devices;
a plurality of wearable devices; and
at least one remote server comprising at least one software platform, an analytics engine, and at least one database;
wherein the plurality of remote devices, the plurality of wearable devices, the plurality of controllable electronic devices, and the at least one remote server are in network communication;
wherein each wearable device of the plurality of wearable devices is designed to capture real-time physiological data of at least one user;
wherein each controllable electronic device of the plurality of controllable electronic devices corresponds to at least one user;
wherein the plurality of remote devices, the plurality of controllable electronic devices, and the plurality of wearable devices are designed to transmit data to the at least one software platform, wherein the plurality of wearable devices transmits the real-time physiological data of at least one user to the at least one remote server, wherein the plurality of controllable electronic devices transmits electronic device data, wherein the electronic device data includes a power status and at least one setting corresponding the plurality of controllable electronic devices;
wherein the analytics engine is designed to determine at least one physiological condition of at least one user based on the real-time physiological data;
wherein based on the at least one determined physiological condition of at least one user, the at least one remote server is operable to generate at least one wellness recommendation based on the at least one determined physiological condition, wherein the at least one wellness recommendation includes at least one action to improve the at least one determined physiological condition; and
wherein the at least one remote server is further operable to transmit an activation command to at least one controllable electronic device of the plurality of controllable electronic devices based on the at least one determined physiological condition and the at least one wellness recommendation.

15. The system of claim 14, wherein the analytics engine is further configured to determine user activity based on at least one wearable device of the plurality of wearable devices, wherein the analytics engine is further operable to transmit at least one nutrition recommendation to at least one remote device based on the user activity, wherein the at least one nutrition recommendation includes consumption of at least one of protein and/or water.

16. The system of claim 14, wherein each wearable device of the plurality of wearable devices includes at least one sensor and a positioning component, wherein the at least one sensor is operable to monitor the physiological condition of the at least one user, wherein the positioning component is operable to track a geographical position of a user, wherein the at least one remote server is operable to modify the at least one wellness recommendation based on the user sensor data and the user position.

17. The system of claim 16, wherein the at least one database further includes property data and location data for a predetermined geographic area, wherein the property data includes building data and amenities data, wherein the amenities data includes a plurality of amenities in the predetermined geographic area, wherein the at least one wellness recommendation further includes at least one amenity of the plurality of amenities in the predetermined geographic area.

18. The system of claim 14, wherein the at least one software platform includes a messaging platform, wherein the messaging platform is operable for electronic communication between each remote device of the plurality of remote devices.

19. The system of claim 14, wherein the at least one wellness recommendation further includes at least one coaching recommendation, wherein the at least one coaching recommendation includes at least one nutrition coach, at least one fitness coach, and/or at least one lifestyle coach, wherein, via the user interface, the software platform is further operable to automatically create an electronic messaging conversation between at least one remote device corresponding to the at least one coaching recommendation and the at least one user.

20. The system of claim 14, wherein each remote device of the plurality of remote devices includes at least one image sensor, wherein the image sensor is designed to capture image data corresponding to at least user, wherein the plurality of remote devices is operable to transmit the user image data to the at least one remote server, wherein the at least one remote server is operable to update the at least one determined physiological condition based on the user image data.
